(19) United States
(12) Patent Application Publication
(10) Pub. No.: US 2025/0339193 A1
(43) Pub. Date: Nov. 6, 2025

(54) APPARATUSES FOR PULSED ELECTRIC FIELD ABLATION THERAPY INCLUDING EXPANDABLE ELECTRODES, AND SYSTEMS AND METHODS THEREOF

(71) Applicant: Alpfa Medical, Inc., Menlo Park, CA (US)

(72) Inventors: Raju VISWANATHAN, Palo Alto, CA (US); Isidro GANDIONCO, Fremont, CA (US)

(21) Appl. No.: 19/213,905

(22) Filed: May 20, 2025

Related U.S. Application Data
(63) Continuation of application No. PCT/US2025/027778, filed on May 5, 2025.
(60) Provisional application No. 63/642,505, filed on May 3, 2024.

(51) Int. Cl. A61B 18/00; A61B 1/012; A61B 1/307; A61B 90/00

(52) U.S. Cl. CPC A61B 18/00; A61B 1/012; A61B 1/307; A61B 90/39; A61B 2018/00077; A61B 2018/00184; A61B 2018/0022; A61B 2018/00267; A61B 2018/00279; A61B 2018/00517; A61B 2018/00577; A61B 2018/00982; A61B 2090/3937

(57) ABSTRACT

In some embodiments, an apparatus comprises a shaft configured to be navigated through an anatomy toward a target tissue of a patient and a plurality of electrodes disposed around a distal portion of the shaft and spaced axially along the shaft. The plurality of electrodes include a distal electrode and a proximal set of electrodes. The distal electrode can be configured to transition from an unexpanded configuration to an expanded configuration independently from the proximal set of electrodes to anchor the distal portion of the shaft relative to the target tissue. The proximal set of electrodes configured to transition from a unexpanded configuration to an expanded configuration to contact neighboring tissue. The plurality of electrodes, after the distal electrode and the proximal set of electrodes are in the expanded configuration, can be configured to deliver pulsed field ablation to ablate at least a portion of the target tissue.

### CROSS-REFERENCE TO RELATED APPLICATIONS

This application is a continuation of PCT Application No. PCT/US2025/027778, filed May 5, 2025, entitled, “APPARATUSES FOR PULSED FOR ELECTRIC FIELD ABLATION THERAPY INCLUDING EXPANDING ELECTRODES, AND SYSTEMS AND METHODS THEREOF,” which claims priority to and the benefit of U.S. Provisional Application No. 63/642,505, filed May 3, 2024, and entitled, “BALLOON CATHETER APPARATUSES AND SYSTEMS FOR PULSED ELECTRIC FIELD ABLATION THERAPY,” the disclosure of each of which is hereby incorporated by reference herein in its entirety.

### TECHNICAL FIELD

This disclosure describes an apparatus and system for delivery of pulsed electric field ablation therapy via interventional access medical procedures.

### BACKGROUND

Ablation in and around tubular anatomies can be challenging since collateral tissue can often be affected by commonly applied ablative therapies such as thermal ablation (RF, cryogenic, or microwave ablation) or laser ablation. There is a need for better approaches to ablation that can selectively act on different tissue types with minimal collateral damage. Pulsed electric field ablation, also known as irreversible electroporation, has been recently developed for cardiac applications as a non-thermal ablation modality. The present disclosure addresses the need for ablation therapy delivery from within a tubular anatomical structure to regions surrounding the tubular structure such as, for example, a vascular structure or the prostatic urethra.

### SUMMARY

In some embodiments, an apparatus includes a shaft configured to be navigated through an anatomy toward a target tissue of a patient; and a plurality of electrodes disposed on a distal portion of the shaft and spaced axially along the shaft, the plurality of electrodes including a distal electrode and a proximal set of electrodes, the distal electrode configured to transition from a unexpanded configuration to an expanded configuration independently from the proximal set of electrodes to anchor the distal portion of the shaft relative to the target tissue, the proximal set of electrodes configured to transition from a unexpanded configuration to an expanded configuration to engage and expand neighboring tissue, the plurality of electrodes, after the distal electrode and the proximal set of electrodes are in the expanded configuration, being configured to deliver pulsed field ablation to the target tissue.

In some embodiments, an apparatus includes a shaft configured be disposed near a target tissue of a patient, the shaft defining a first lumen and a second lumen along a length thereof; a first electrode including a first conductive element and a first expandable element, the first lumen configured to convey fluid to the first expandable element to expand the first expandable element such that the first conductive element transitions from an unexpanded configuration to an expanded configuration; and a second electrode including a second conductive element and a second expandable element, the second lumen configured to provide fluid to expand the second expandable element such that the second conductive element transitions from an unexpanded configuration to an expanded configuration, the first electrode and the second electrode in the expanded configuration configured engage and expand neighboring tissue and to deliver pulsed field ablation to the target tissue.

In some embodiments, an apparatus includes a shaft configured to be navigated through a working channel of a cystoscope to a urethra of a patient; a plurality of electrodes spaced axially along a distal portion of the shaft; and a plurality of markers, each marker from the plurality of markers disposed at a proximal end of a respective electrode from the plurality of electrodes to indicate when the respective electrode is disposed distal to a distal end of the cystoscope, the plurality of electrodes configured to transition from an unexpanded configuration to an expanded configuration in which the plurality of electrodes are configured to engage and expand at least a wall of the urethra and deliver pulsed field ablation energy via at least a portion of the urethra to prostate tissue adjacent thereto.

In some embodiments, a method includes navigating a shaft to an anatomy of a patient toward a target tissue, the shaft including a plurality of electrodes disposed on a distal portion of the shaft and spaced axially along the shaft; expanding a distal electrode of the plurality of electrodes against a portion of tissue near the target tissue to anchor the distal portion of the shaft relative to the target tissue; expanding, after expanding the distal electrode, a proximal set of electrodes of the plurality of electrodes; and applying, after the distal electrode and the proximal set of electrodes are expanded, pulsed field ablation to at least a portion of the target tissue via the plurality of electrodes.

In some embodiments, a method includes navigating a shaft to a urethra of a patient adjacent to a bladder; expanding a distal electrode disposed on a distal portion of the shaft to engage and expand at least a portion of a bladder neck or a proximal portion of the bladder to anchor the distal portion of the shaft relative to a prostate; expanding, after expanding the distal electrode, a proximal electrode disposed on the distal portion of the shaft to engage and expand a wall of the urethra; and applying, when the distal electrode and the proximal electrode are expanded, pulsed field ablation via the first electrode and the second electrode to at least a portion of the prostate.

In some embodiments, a method includes advancing a cystoscope or similar visualizing scope through an anatomy of a patient to a target site; advancing a shaft through a working channel of the cystoscope until a first marker associated with a first electrode disposed on a distal portion of the shaft is visible in an image of the cystoscope, indicative of the first electrode being disposed distal to a distal end of the cystoscope; transitioning, after the first marker is visible in the image, the first electrode from an unexpanded configuration to an expanded configuration; withdrawing the cystoscope proximally until a second marker associated with a second electrode disposed on the distal portion of the shaft is visible in the image, indicative of the second electrode being disposed distal to the distal end of the cystoscope; transitioning, after the second marker is visible in the image, the second electrode from a unexpanded configuration to an expanded configuration; and applying, via the first electrode and the second electrode, pulsed field ablation to at least a portion of the target tissue.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic block diagram of a cystoscope including a working channel and an imaging element, according to embodiments.

FIG. 1B is a schematic block diagram of a catheter for delivery pulsed field ablation, according to embodiments.

FIGS. 2A-2C illustrate a catheter assembly for delivering pulsed field ablation to a target tissue, according to embodiments.

FIG. 3 illustrates an example cylindrical braid of a catheter for delivering pulsed field ablation, according to embodiments.

FIG. 4A illustrates an example cylindrical braid of a catheter for delivering pulsed field ablation held in a modified shape, according to embodiments.

FIG. 4B illustrates an example cylindrical braid of a catheter for delivering pulsed field ablation held in a modified shape, according to embodiments.

FIG. 5 illustrates a distal portion of an example catheter of the present disclosure with a shaft and three expandable electrodes in an unexpanded configuration, according to embodiments.

FIG. 6 illustrates a distal portion of an example catheter of the present disclosure with a shaft and three expandable electrodes in an expanded configuration, according to embodiments.

FIG. 7 illustrates an example catheter of the present disclosure, showing electrical leads connecting to electrodes and lumens for balloon inflation to expand the electrodes, according to embodiments.

FIG. 8 provides a schematic illustration of an example catheter device of the present disclosure used in conjunction with a cystoscope to access the prostatic urethra, according to embodiments.

FIG. 9 shows an example catheter inserted through a working channel of a cystoscope in a first configuration, according to embodiments.

FIG. 10 depicts an example catheter inserted through a working channel of a cystoscope in a second configuration, according to embodiments.

FIG. 11 illustrates an example catheter inserted through a working channel of a cystoscope in a third configuration, according to embodiments.

FIG. 12 schematically illustrates an example catheter of the present disclosure including a proximal portion thereof, according to embodiments.

FIG. 13 provides a schematic illustration of a proximal section of an example catheter of the present disclosure showing markers on the proximal portion of a catheter shaft, according to some embodiments.

FIG. 14 provides a schematic illustration of an example catheter of the present disclosure that is passed through a working channel of a cystoscope showing two electrodes exposed outside a distal end of the cystoscope, according to embodiments.

FIG. 15 provides an illustration of an example catheter with four expandable electrodes, with all electrodes in an undeployed state, according to embodiments.

FIG. 16 illustrates an example catheter with four expandable electrodes with the distal electrode in a deployed or expanded state and the proximal three electrodes in an undeployed state, according to embodiments.

FIG. 17 illustrates an example catheter with four expandable electrodes with all four electrodes expanded by inflation of corresponding balloons, according to embodiments.

FIGS. 18A-18B illustrates an example catheter including a plurality of electrodes and a plurality of markers each disposed proximally respectively to each of the plurality of electrodes, according to embodiments.

FIG. 19 illustrates an example cystoscope configured to receive a catheter therethrough for delivering pulsed field ablation device in a patient, according to an embodiment.

FIGS. 20A-20F show a method of positioning electrodes relative to an urethra to deliver ablative energy to a prostate, according to an embodiment.

FIGS. 21A-21H show cystoscopic images of a procedure for delivering pulsed field ablation to a prostate.

FIG. 22 illustrates electrodes at a distal end of a catheter shaft partially disposed in a bladder, according to embodiments.

FIG. 23 a is flow chart of an example method of delivering ablative energy to a target tissue, according to embodiments.

FIG. 24 is a flow chart of an example method of delivering ablative energy to a prostate, according to embodiments.

### DETAILED DESCRIPTION

This disclosure details catheter structures for pulsed electric field ablation delivery with electrodes in the form of expandable structures or elements that can be activated to change shape (e.g., to an expanded configuration, inflated configuration, or deployed configuration) and deactivated to recover their original shape (e.g., an unexpanded configuration, elongated configuration, deflated configuration, collapsed configuration, or undeployed configuration). In some embodiments, the expandable structures can be activated or expanded by inflation with a fluid. In some embodiments, the electrodes can include a cage-like metallic structure such as, for example, braided constructions or patterned constructions with struts that can be cut from tubular metallic shapes. The metallic structures generally can include a shape memory or superelastic material such as, for example, Nitinol alloy.

Embodiments described herein can enable delivery of pulsed field ablation with reliable positioning. The embodiments described herein can include a distal electrode configured to independently expand against an anchor location to position the distal end of a catheter relative to a target tissue.

Embodiments described herein can enable delivery of pulsed field ablation to a wide range of anatomies. For example, the devices described herein can access different prostate anatomies (e.g., anatomies with enlarged median lobes, different prostate sizes, etc.) and/or other tubular anatomies. The embodiments described herein can provide an easy to implement and low risk procedure, such as for benign prostatic hyperplasia (BPH), with reduced procedural complications and improved patient outcomes. For example, the methods described herein may reduce post-procedure healing time, reduce risk of hematuria, eliminate need for a catheter post-procedure, and preserve ejaculatory function, and treat BPH symptoms without use of an implant. The embodiments described herein can be carried out in a clinical setting (e.g., hospitals, doctor's offices, ambulatory surgical centers (ASC)).

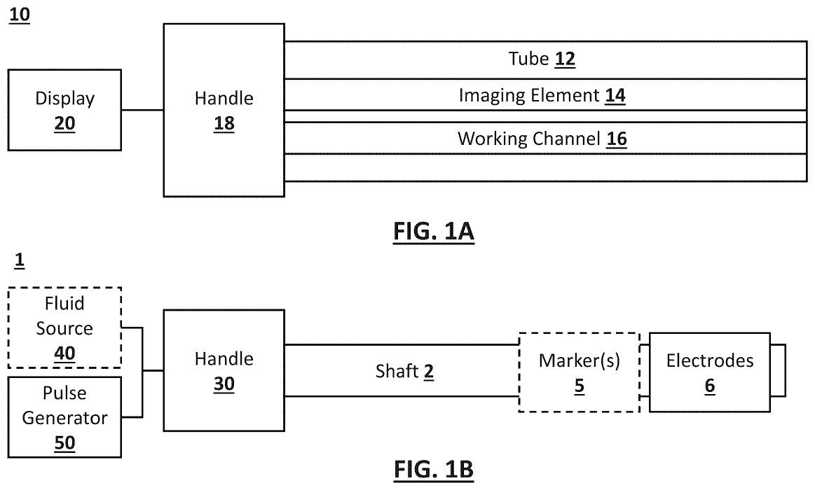

FIG. 1A is a schematic block diagram of a cystoscope 10 including a tube 12 (e.g., an insertion tube, cystoscope shaft) configured to navigate through an anatomy (e.g., a body lumen or other tubular structure) of a patient to a target tissue. The tube 12 may be coupled to a handle 18 at a proximal end thereof. As shown, the tube 12 of the cystoscope 10 may define a working channel 16 and be configured to receive a catheter (e.g., catheter 1 shown in FIG. 1B, or any of the other catheters and devices described herein) therethrough. The cystoscope 10 can further include an imaging element 14 configured to image anatomy near a distal end of the cystoscope 10, e.g., to guide navigation and positioning of the catheter. In some embodiments, the imaging element 14 may include a lens positioned at a distal end of the tube 12 and an optical fiber extending along a length of the tube 12. In some embodiments, the imaging element 14 may include a sensor or camera (e.g., a Complementary Metal-Oxide Semiconductor (CMOS) camera) at a distal end of the tube 12, which can be coupled via a wired or wireless connection to a display or other compute device (e.g., processor). For example, in some embodiments, the camera can be coupled via an electrical wire that extends along the tube 12 of the cystoscope to a proximal end of the cystoscope 10, e.g., for coupling to a compute device or display. In some embodiments, the imaging element 14 may be coupled to a display 20 (e.g., via one or more ports disposed in the handle 18) and be configured to display image data collected by the imaging element 14 to an user during the procedure. The handle 18 can be configured to be engaged by an user to navigate the tube 12 through the anatomy. In some embodiments, the handle 18 may define an entry port (e.g., a working channel port) configured to receive the catheter (e.g., catheter 1 depicted in FIG. 1B) such that the catheter can be advanced through the working channel 16. In some embodiments, the cystoscope 10 may be configured for insertion through urethra, while in other embodiments it can be a variant device such as an endoscope or other visualizing scope intended for access to other tubular structures, for example, a portion of the digestive tract.

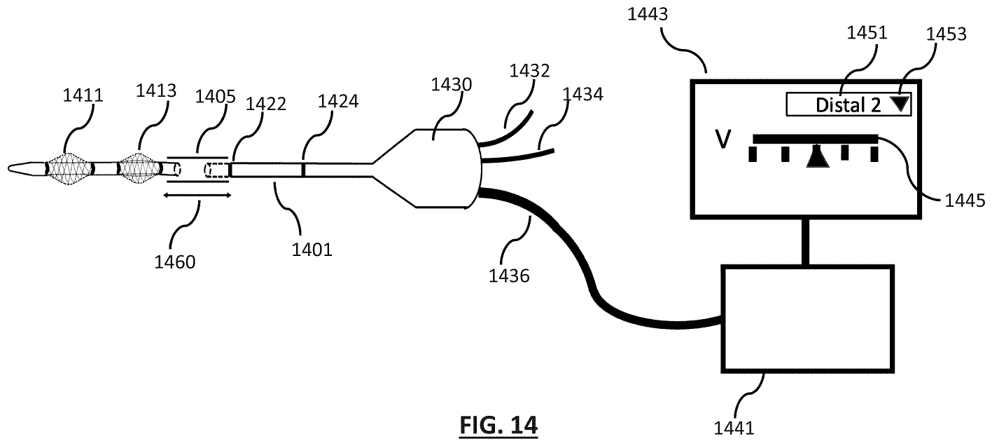

FIG. 1B is a schematic block diagram of a catheter 1 configured to deliver pulsed field ablation to a target tissue of a subject or patient, according to embodiments. As shown, the catheter 1 may include a shaft 2 including a plurality of electrodes 6 disposed on a distal portion thereof. In some embodiments, the electrodes 6 may be axially spaced along the distal portion of the shaft. The electrodes 6 can be configured to apply pulsed field ablation to the target tissue. The distal portion of the shaft 2 can include any suitable number of electrodes 6 such as at least 2 electrodes, at least 3 electrodes, at least 4 electrodes, at least 5 electrodes. In some embodiments, the distal portion of the shaft 2 may include between 2 electrodes and 4 electrodes, inclusive of all ranges and subranges therebetween, depending on the anatomy of the patient. In some embodiments, the electrode(s) 6 may define a predetermined spacing or distance therebetween, described in further detail in FIG. 14. In some embodiments, adjacent electrode 6 pairs may define equivalent spacing therebetween. In some embodiments, one or more adjacent electrode 6 pairs may define variable spacing therebetween. As described below, the electrodes 6 can include one subset of electrodes that can be polarized with one electrical polarity and a second subset of electrodes that can be polarized with the opposite electrical polarity, to deliver irreversible electroporation or pulsed field ablation.

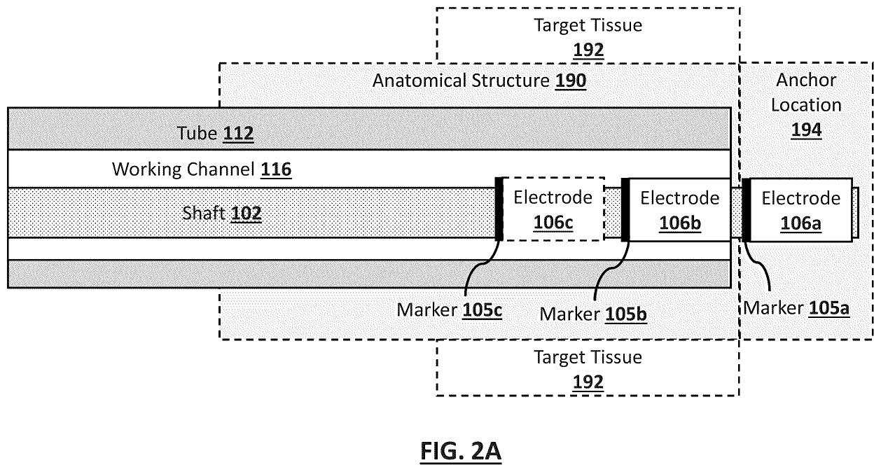

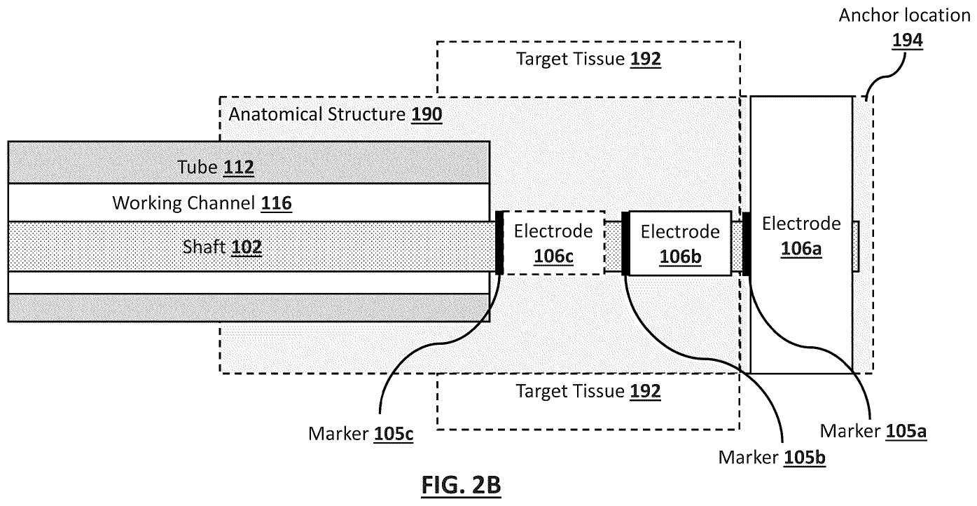

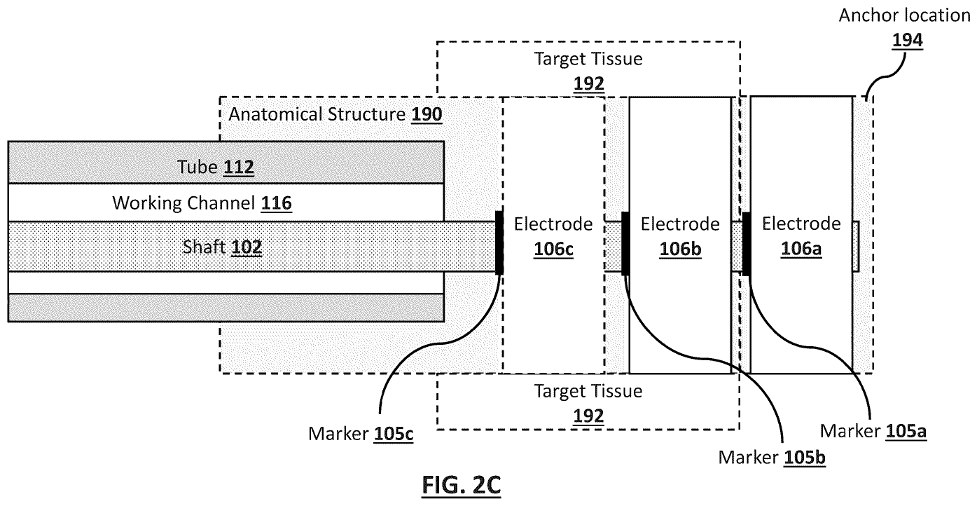

Optionally, the shaft 2 can further include one or more markers 5 disposed on the distal portion of the shaft. In some embodiments, a marker 5 can be disposed adjacent to each electrode 6, e.g., to indicate a location of that electrode 6. More specifically, the markers 5 may be disposed at a proximal end of one or more of the electrodes 6 to indicate to the user when a full length of the electrode 6 is disposed distal to a distal end of the cystoscope 10, when the distal portion of the shaft 2 is extended out of the working channel 16 of the cystoscope 10. This is described in further detail with respect to FIGS. 2A-2C. In some embodiments, the markers 5 may include any suitable markers such as, for example, colored markers, radiopaque markers, indentations or raised portions of the shaft, reflective markers, etc.

In some embodiments, the electrodes 6 can be expandable and configured to transition between an unexpanded configuration (e.g., elongated, collapsed, deflated, undeployed, etc.) and an expanded configuration (e.g., inflated, expanded, deployed, etc.). In some embodiments, the electrodes 6 can be configured to expand in response to inflation and/or by mechanical actuation including fluid-driven actuation (e.g., via actuation of a button, lever, or other actuator disposed on a handle 30 of the catheter 1 that in embodiments can include inflation or deflation of the electrode(s) with a fluid-filled syringe). In some embodiments, the electrodes 6 can be self-expanding (e.g., the electrodes 6 may be memory set to expand to an expanded configuration) after being disposed outside of the working channel 16 of the cystoscope 10.

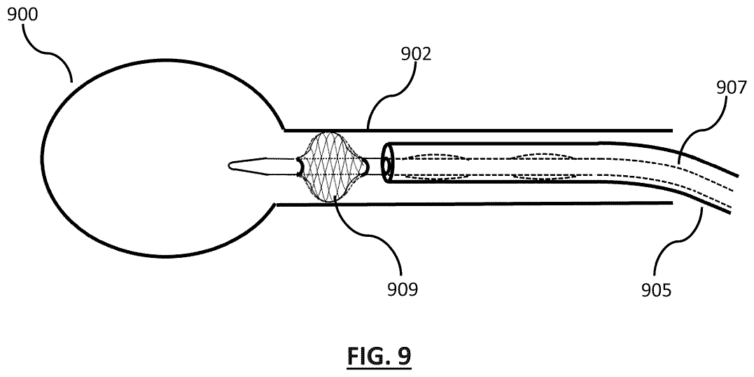

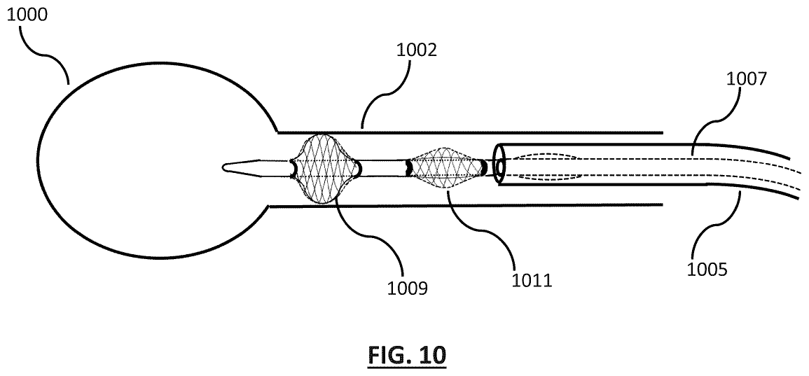

In some embodiments, the electrodes 6 can include a first electrode and a second electrode proximal to the first electrode. In some embodiments, the electrodes 6 can include a first electrode and a set of electrodes proximal to the first electrode. In some embodiments, the first electrode (e.g., the distal electrode) may be configured to transition from an unexpanded configuration to an expanded configuration independently from of the more proximally situated electrodes (e.g., the proximal electrode, the proximal set of electrodes). The first electrode can be configured to expand to engage a portion of patient anatomy, e.g., to anchor the distal portion of the shaft 2 of the catheter 1 in the anatomy, as described in FIGS. 9-10. For example, the distal electrode may be configured to engage an inner surface of a body lumen to anchor the distal portion of the shaft relative to the target tissue (e.g., the wall of the urethra). The distal electrode in the expanded configuration may be configured to engage and expand at least one of the wall of the urethra, a bladder neck, and/or a proximal portion of the bladder. In some embodiments, the proximal electrode or the proximal set of electrodes can be configured to expand (e.g., after the distal portion of the shaft is anchored via the expansion of the first electrode) to engage and expand a portion of the body lumen proximal to the distal electrode. In some embodiments, the electrodes 6 in the expanded configuration may be configured to apply a predetermined pressure to the body lumen (e.g., the urethra) sufficient to expand the body lumen. The proximal electrode or the proximal set of electrodes may be configured to transition (e.g., collectively) from an unexpanded configuration to an expanded configuration to engage and expand the wall of the urethra. The electrodes 6 in the expanded configuration may be configured to press against the wall of the urethra to expand or open up the urethra before delivery of pulsed field ablation. In some embodiments, the electrodes 6 may be configured to deliver pulsed field ablation to neighboring tissue, including, for example, prostate tissue (e.g., the target tissue). In some embodiments, the electrodes 6 can be configured to deliver pulsed field ablation to the neighboring tissue once the body lumen has been expanded to a predetermined diameter.

In some embodiments, after delivery of pulsed field ablation, the electrodes 6 may be transitioned to the unexpanded configuration and repositioned to a new location relative to the target tissue. The electrodes 6 can then be expanded against a new portion of the anatomy (e.g., a new portion of the urethra such as a portion more proximal to the portion that was previously ablated). In some embodiments, with each repositioning, the distal electrode may be independently expanded to anchor the distal end of the shaft 2 in the patient anatomical structure. While the electrode being used to anchor the distal portion of the shaft 2 is described as being the distal electrode, it can be appreciated that in some embodiments, the electrode configured to anchor the distal portion of the shaft is located between other electrodes, proximal to other electrode(s) and/or otherwise situated along a length of the shaft. In some embodiments, more than one electrode may be configured to anchor against the anatomy.

In some embodiments, each electrode 6 may include an electrically conductive element. The conductive elements can include one or more conductive strands (e.g., metallic and/or metal-alloy strands). In some embodiments. the conductive strands can be formed or woven into a braid, cage-like, and/or mesh structure. In some embodiments, the conductive element can form a cylindrical braid configured to be deformed (e.g., longitudinally and/or radially) to transition between the unexpanded configuration and the expanded configuration. For example, the cylindrical braid may be configured to expand in response to an outward radial force or pressure applied to the cylindrical braid.

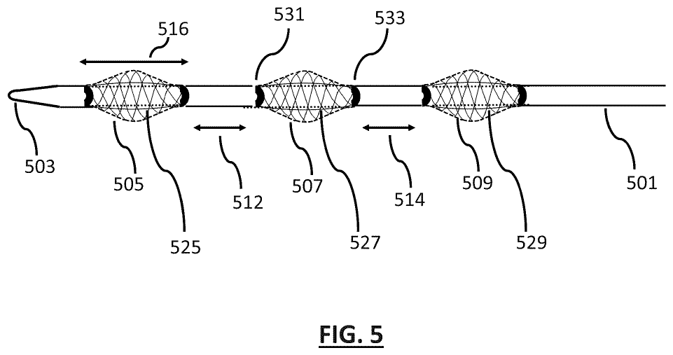

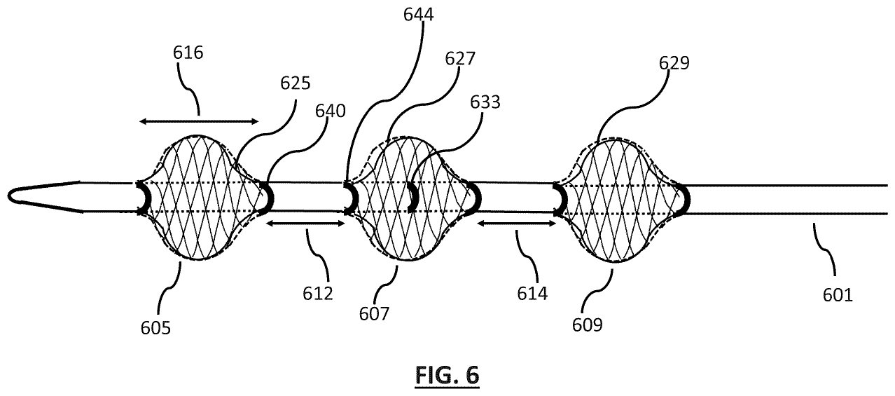

In some embodiments, each electrode 6 can include an expandable element. The expandable element may be configured to apply a force to or to deform/expand the conductive element such that the electrode 6 transitions from the unexpanded configuration and the expanded configuration. In some embodiments, the electrode 6 can transition from the expanded configuration to the unexpanded configuration when the expandable element stops applying the force to the conductive element. In some embodiments, each electrode 6 may be disposed around the distal portion of the shaft 2 with the expandable element being disposed underneath at least a portion of the conductive element. For example, the cylindrical braid may be disposed around the expandable element and the shaft 2 (e.g., shown in FIGS. 5-6). In some embodiments, the expandable elements may include an inflatable structure such as a balloon.

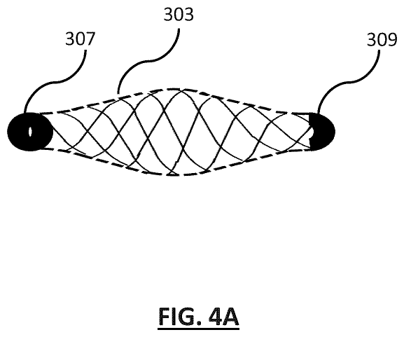

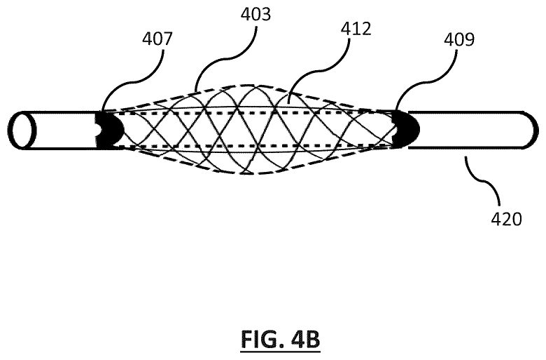

Each electrode 6 may include a first sleeve at a first end of the conductive element and a second sleeve at a second end of the conductive element to couple the conductive element to the shaft 2, described in further detail in FIG. 4B. In some embodiments, a first end of the conductive element (e.g., the cylindrical braid) may be fixed to the shaft 2 and a second end of the conductive element may be free to move along the shaft 2. For example, the proximal end of the conductive element may be fixed to the shaft, and the distal end of the conductive element may be free to move along the shaft 2. Therefore, when the expandable element expands and applies a force to the conductive element, the free or unfixed end can move along the shaft 2 such that a total length of the conductive element decreases while a diameter of the conductive element increases, described in further detail in FIGS. 5-6.

In some embodiments, the shaft 2 can define one or more lumens (not shown) in fluid communication with the expandable elements of the electrodes 6. For example, the one or more lumens can be configured to convey fluid from a fluid source 40 through the shaft 2 and into the expandable elements. In some embodiments, the shaft 2 may define one or more openings in the distal portion configured to place the one or more lumens in fluid communication with the expandable elements of the electrodes 6. For example, each electrode 6 can be configured to align with a respective set of openings defined in the shaft 2.

In some embodiments, the shaft 2 can define a first lumen configured to convey fluid from the fluid source 40 to the first electrode. More specifically, the first lumen can be configured to convey fluid to a first expandable element of the first electrode to expand the first expandable element such that a first conductive element of the first electrode transitions from the unexpanded configuration to the expanded configuration. The shaft 2 can define a second lumen configured to convey fluid from the fluid source 40 to the second electrode (or set of electrodes). For example, the second lumen can convey fluid to a second expandable element of the second electrode to expand the second expandable element such that a second conductive element of the second electrode transitions from the unexpanded configuration to the expanded configuration. Therefore, the first electrode can be actuated via the first lumen and the second electrode can be actuated via the second lumen independently. In some embodiments, the second lumen may be configured to convey fluid to the proximal set of electrodes. For example, the second lumen may terminate in a set of openings aligned with the proximal set of electrodes such that the proximal set of electrodes can be collectively transitioned to the unexpanded configuration to the expanded configuration. In some embodiments, the shaft 2 may define a plurality of lumens and each electrode 6 may be in fluid communication with a respective lumen such that each electrode 6 can be expanded independently. In some embodiments, any subset of electrodes of the plurality of electrodes 6 may be configured to be expanded collectively.

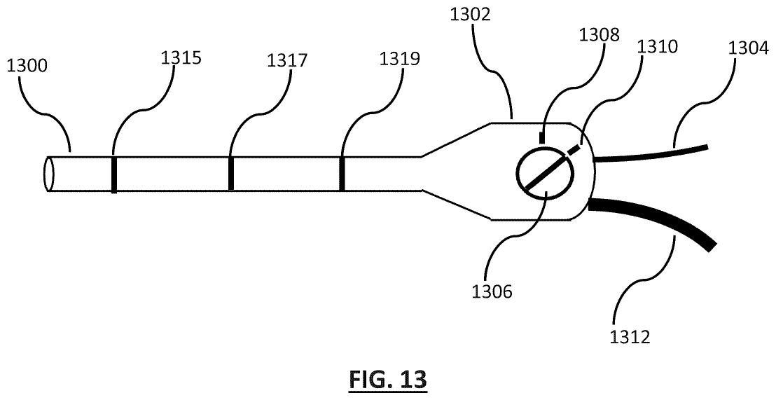

In some embodiments, the proximal end of the shaft 2 may be coupled to a handle 30. In some embodiments, the handle 30 can be configured to be engaged by the user to advance or retract the catheter 1 relative to the cystoscope 10. In some embodiments, the handle 30 can be couple the shaft 2 to the fluid source 40. In some embodiments, the handle 30 may include one or more flow control mechanisms configured to control fluid flow from the fluid source 40) through the lumens of the shaft 2. In some embodiments, the handle 30 may include one or more actuators configured to be engaged by the user to control expansion of the electrodes 6 (e.g., by controlling inflation of the expandable members with a syringe), also described in FIG. 13. In some embodiments, the handle 30 may be configured to allow fluid flow through the first lumen while preventing fluid flow through the second lumen such that the first electrode can transition to the expanded configuration separately from the second electrode (or set of electrodes). For example, the handle 30 may include a flow control mechanism having a first configuration in which the flow control mechanism places the first lumen in fluid communication with the fluid source 40 such that fluid can flow through the first lumen to transition the first electrode to the expanded configuration, while preventing fluid flow through the second lumen such that the second electrode remains in the unexpanded configuration. Additionally, the fluid control mechanism may have a second configuration in which the flow control mechanism places the second lumen in fluid communication with the fluid source 40) (e.g., after the first electrode is expanded) such that fluid can flow through the second lumen to transition the second electrode to the expanded configuration. In some embodiments, the electrodes 6 can be expanded to a predetermined radius and/or to apply a predetermined force on neighboring tissue based on fluid flow into the expandable elements. In some embodiments, the user can control expansion of the electrodes 6 using the actuator of the handle 30. In some embodiments, the shaft 2 may be coupled to the fluid source 40 directly. For example, a proximal end of the shaft 2 may include one or more ports (e.g., valves or Luer locks) configured to couple to the fluid source 40.

In some embodiments, the shaft 2 may include one or more electrical conductors (e.g., a wire) (not shown) configured to extend along a length of the shaft 2 to couple the electrode(s) 6 to a pulse generator 50. In some embodiments, the conductor may be insulated along the length of the shaft 2, as described in further detail in FIG. 5. The pulse generator 50 may be configured to supply a pulsed waveform to the electrodes 6 such that the electrodes 6 can deliver the pulsed field ablation to the target tissue. In some embodiments, the handle 30 may be configured to couple the pulse generator 50 to the shaft 2. In some embodiments, the shaft 2 may be coupled to the pulse generator 50 directly. For example, a proximal end of the shaft 2 may include one or more electrical connectors configured to connect to the pulse generator 50.

FIGS. 2A-2C illustrate steps of using a system for delivering pulsed field ablation to a target tissue 192. As shown, a distal end of a tube 112 of a cystoscope (e.g., cystoscope 10 as described above) can be navigated to an anatomical structure 190 near a target tissue 192. The tube 112 can define a working channel 116 through which a shaft 102 of a catheter can be disposed. In some embodiments, the shaft 102 can include a first electrode 106a, a second electrode 106b, and optionally a third electrode 106c disposed on a distal portion of the shaft and axially spaced along the distal portion of the shaft 102. The shaft 102 can further include a plurality of markers 105a, 105b, 105c disposed behind or at a proximal end of each of the respective electrodes 106a-106c. In some embodiments, shaft 102 can include a first marker 105a disposed at a proximal end of the first electrode 106a and a second marker 105b disposed at a proximal end of the second electrode 106b. In some embodiments, the shaft 102 may optionally include a third marker 105c disposed at a proximal end of the third electrode 106c. In some embodiments, the proximal end of each electrode 106a-106c and the markers 105a-105c may be fixed relative to each other and relative to the shaft 102. In some embodiments, the catheter including shaft 102, electrodes 106a, 106b, and 106c, and markers 105a, 105b, 105c can be structurally and/or functionally similar to the catheter 1 including the shaft 2, electrodes 6, and markers 5, and therefore, certain details of the catheter are not described herein with respect to FIGS. 2A-2C.

In some embodiments, the tube 112 can be positioned such that the distal end of the tube 112 is proximal (e.g., immediately proximal) to an anchor location 194 (e.g., based on images captured by the cystoscope). In some embodiments, the shaft 102 can be configured to be advanced distally to position the first electrode 106a adjacent to the anchor location 194, as shown in FIG. 2A. In some embodiments, the marker 105a can be configured such that the marker 105a is visible on images captured by the cystoscope to indicate when a full length of the electrode 106a is distal to the distal end of the tube 112. In some embodiments, the first electrode 106a can be advanced while in an unexpanded configuration. After the electrode 106a is positioned adjacent to the anchor location 194 (e.g., when the first marker 105a is visible). the first electrode 105a can be transitioned from the unexpanded configuration to the expanded configuration to expand against the anchor location 194 and anchor the distal portion of the shaft 102 relative to the target tissue 192, as shown in FIG. 2B. After the first electrode 106a transitions to the expanded configuration and/or the distal portion of the shaft 102 has been anchored relative to the target tissue 192, the tube 112 can be withdrawn proximally until the second electrode 106b (and optionally the third electrode 106c) is disposed distal to the distal end of the tube 112. In some embodiments, the second marker 105b when visible on the cystoscope images can indicate to the user that a full length of the second electrode 106 is disposed distal to the tube 112. Similarly, the third marker 105c may indicate when all three electrodes 106a-106c are disposed distal to the distal end of the tube 112. In some embodiments, the first marker 105a and the second marker 105b may be different markers (e.g., different colors, lengths, brightness, etc.) such that the user can differentiate between the first electrode 106a and the second electrode 106b. In some embodiments, the first marker 105a may be a unique marker that is different than the markers proximal to the first marker 105a. In some embodiments, the electrode configured to expand independently from the other electrodes may include the unique marker.

In some embodiments, the tube or cystoscope 112 can be withdrawn until each of the electrodes 106a-106c are disposed distal to the distal end of the tube 112 (e.g., the electrodes 106a-106c are exposed in the anatomical structure 190). After the second electrode 106b, and optionally the third electrode 106c, are disposed distal to the tube 112, the second electrode 106b, and optionally the third electrode 106c, can be transitioned to the expanded configuration to engage and expand the anatomical structure 190 adjacent to the target tissue 192, as shown in FIG. 2C. Once the electrodes 106a-106c are in the expanded configuration, the electrodes 106a-106c can be activated to deliver pulsed field ablation to surrounding tissue including the target tissue 192. In some embodiments, the electrodes 106a-106c can be transitioned back to the unexpanded configuration, repositioned (e.g., moved proximally), and transitioned to the expanded configuration such that a second portion of the target tissue can be ablated. In some embodiments, the anatomical structure 190) can be a prostatic urethra, and the tube 112 can be navigated through the prostatic urethra to a desired placement with respect to prostate tissue. In some embodiments, the anchor location can include any one of a distal portion of the wall of the urethra, a bladder neck, and/or a proximal portion of the bladder.

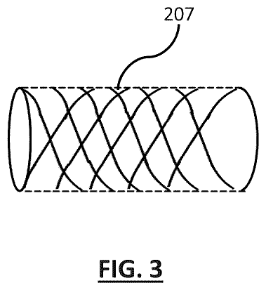

FIG. 3 illustrates a cylindrical braid 207, according to embodiments. The cylindrical braid 207 can be a braided construction including metallic wire strands that cross each other in a braiding pattern that provides structural stability. In some embodiments, the wire material can include a superelastic material such as Nitinol, after heat treatment according to known methods, the cylindrical structure can recover its structural shape even after a significant deformation. Furthermore, when held in a stressed state to achieve a modified shape and subsequently deformed by additional applied forces, the modified shape can be recovered when the additional applied forces are removed.

FIG. 4A illustrates an originally cylindrical braid comprising metallic wire strands held in a modified shape 303 (e.g., modified from cylindrical), according to embodiments. The cylindrical braid depicted in FIG. 4 can be structurally and/or functionally similar to other cylindrical braids described herein, including, for example the cylindrical braid described with reference to FIG. 3. The cylindrical braid can be held in the modified shape 303 by stretching the cylindrical braid and holding down its ends via attachment to sleeves such as 307 and 309 at each end. In the modified shape, the cylindrical braid may include a larger central portion having a first diameter that narrows or tapers to a second diameter smaller than the first diameter near each end of the cylindrical braid. The sleeves 307 and 309 can comprise one or more layers of polymeric material such as, for example, polyimide or Pebax. In embodiments, an adhesive (e.g., glue) and/or heat bonding can be used to attach the sleeves 307 and 309 to the cylindrical braid, with assembly performed over a mandrel. In embodiments, the sleeves 307 and 309 can comprise a metallic ring to which the braid is attached or welded. For example, laser welding can be employed to attach such a metallic ring to the braid. In embodiments, additional polymeric sleeves and/or heat shrink tubing can be attached to the metallic ring for attachment or bonding to a catheter shaft. The original cylindrical braid can comprise a superelastic material, such as, for example. Nitinol, with the cylindrical braid being heat treated according to methods known in the art to form a “memory” of the shape. When the modified shape 303 (e.g., modified from cylindrical) of the braid is subsequently deformed by additional applied forces, the deformed shape (e.g., expanded, compressed, enlarged, etc.) can recover back to its modified shape (e.g., elongated with tapered ends) when the additional applied forces are removed.

FIG. 4B illustrates an originally cylindrical braid including metallic wire strands held in a modified shape 403, according to embodiments. The cylindrical braid depicted in FIG. 4B can be structurally and/or functionally similar to other cylindrical braids described herein, including, for example the cylindrical braids described with reference to FIGS. 3 and 4A. The cylindrical braid can be held in the modified shape 403 by stretching the cylindrical braid and holding down the ends of the cylindrical braid via attachment to a sleeve such as a first end sleeve 407 and a second end sleeve 409 at each end of the cylindrical braid. For example, an edge of each end of the cylindrical braid may be radially compressed by the sleeve to form the modified shape. The originally cylindrical braid can comprise a superelastic material such as, for example, Nitinol, the cylindrical braid can be heat treated according to methods known in the art to form a “memory” of the shape. In embodiments, a balloon 412 (or any suitable inflatable element) with a generally tubular aspect is placed over a shaft 420 and the balloon ends are attached to the shaft. The balloon 412 can comprise a material such as, for example, silicone, polyurethane, nylon, polyethylene or other polymer materials with a thin wall that are employed in the medical device industry. The shaft 420 can also comprise polymeric material such as, for example, Pebax or nylon, and the balloon 412 can be attached to the shaft 420 with glue or by forming a heat bond. One of the end sleeves 407, 409 of the braid 403 can be attached to one end portion of the balloon 412 over the shaft 420. For example, the second end sleeve 409 can be attached to one end portion of the balloon 412 over the shaft 420. In embodiments, one or more layers of heat shrink tubing can be utilized over the attached end sleeve 409 to hold the attached sleeve 409 down on the shaft. The other of the end sleeves can be free to slide over the shaft. For example, the first end sleeve 407 can be free to slide over the shaft 420. The length of the braid 403 including the end sleeves 407, 409 can be longer than the length of the balloon 412 that is not attached to the shaft.

In embodiments, at least one of the end sleeves 407 or 409 can comprise a metallic ring to which the braid is attached or welded. For example, laser welding can be employed to attach such a metallic ring to the braid. In embodiments, additional polymeric sleeves and/or heat shrink tubing can be attached to the metallic ring for attachment or bonding to the shaft 420. The diameter of the shaft 420 can be smaller than the diameter of the braid in its cylindrical form. In embodiments, the diameter of the shaft 420 is at least about 20% smaller than the diameter of the braid in its cylindrical form. In some embodiments, the diameter of the shaft 420) is at least about 10% smaller, about 15% smaller, about 20% smaller, about 25% smaller, about 30% smaller, about 35% smaller than the diameter of the braid in cylindrical form. With the balloon 412 mounted on the shaft 420, the shaft 420) can have one or more holes (not shown) underneath the balloon 412 with the hole(s) exiting an internal or inner lumen (not shown) in the shaft 420, e.g., for inflation of the balloon 412 with a fluid. In some embodiments, the one or more balloons may be aligned with the balloon 412 along a length of the shaft and in fluid communication with the balloon 412. In some embodiments, the shaft may define a lumen therethrough configured to allow a flow of fluid to flow through the one or more holes and into the balloon 412 to inflate the balloon. When the balloon 412 is inflated in this manner, the braid 403 expands and the end sleeve (e.g., end sleeve 407 in the example above) that is free to move can slide toward the other fixed or attached end sleeve (e.g., end sleeve 409 in the example above) to shorten the effective length of the braid while the diameter of the braid 403 increases. When the braid 403 is attached to appropriate electrical leads, the braid can be configured as an expandable electrode. Subsequent to inflation, a vacuum or suction force can be applied through the lumen of the shaft to withdraw fluid from the balloon 412, thereby deflating the balloon 412. When the balloon deflates, the uninflated or unexpanded shape of the braid can be recovered (e.g., shown in FIG. 4B), and the free end sleeve can slide back to its original position.

FIG. 5 illustrates a distal portion of an example catheter of the present disclosure, according to embodiments. The catheter can include a shaft 501, an atraumatic distal tip 503, and three expandable electrodes 505, 507 and 509. The expandable electrodes 505, 507 and 509 can each be formed from or include a cylindrical braid, such as those described with reference to FIGS. 3-4B. Each expandable electrode 505, 507 and 509, respectively, can include an inflatable balloon 525, 527 and 529 disposed underneath the braid and over the corresponding shaft portions. The expandable electrodes 505, 507 and 509 and the balloons 525, 527 and 529 can be structurally and/or functionally similar to the cylindrical braids and balloons described in other embodiments, e.g., including the embodiment in FIG. 4B above. Each electrode 505, 507 and 509 can have a proximal end sleeve that is fixed or attached to the catheter shaft 501 and a distal end sleeve that is free to slide along the shaft 501, as described with reference to FIG. 4B. For example, electrode 507 has a distal end sleeve 531 that is free to slide over the shaft and a proximal end sleeve 533 that is fixed to the catheter shaft 501. In other embodiments, the distal end sleeve of the electrodes 505, 507 509 can be fixed or attached to the catheter shaft 501 and the proximal end sleeve can be free to slide along the shaft 501. In some embodiments, each braid is connected electrically to an electrical lead wire. For example, the connection or attachment of the electrical lead to the cylindrical braid can be at the proximal portion of the braid in the sleeve portion. In some embodiments, the electrical lead wire can be insulated over the major portion of its length, e.g., with a high dielectric strength material that can withstand a voltage of at least approximately 500 Volts across its thickness without dielectric breakdown. In this manner, each electrode can be configured to deliver high voltage pulses to an anatomy of interest. In the unexpanded configuration shown in FIG. 5, each braid has an associated length 516. In this configuration, electrodes 505 and 507 are separated by a spacing 512, and electrodes 507 and 509 are separated by a spacing 514. The spacing 512 can be equal to or different from the spacing 514. The shaft portion of each balloon, such as for example 525, can have one or more holes (not shown) underneath the balloon with the hole(s) exiting an internal or inner lumen (not shown) in the shaft for inflation of the balloon with a fluid.

FIG. 6 illustrates a distal portion of an example catheter of the present disclosure with a shaft 601 and showing three expandable electrodes 605, 607 and 609. The catheter of FIG. 6 can be structurally and/or functionally similar to other catheters disclosed herein, including, for example, the catheter described with reference to FIG. 5. Each expandable electrode or braid 605, 607 and 609, respectively, has an inflatable balloon 625, 627 and 629 disposed underneath the braid and over the corresponding shaft portions. As described in the foregoing figures, each inflatable balloon 625, 627, 629 can be inflated by infusing fluid into the inflatable balloon 625, 627, 629 via appropriate holes in the catheter shaft 601 underneath each balloon 625, 627, 629. In some embodiments, the shaft 601 may define or include one or more lumens in fluid communication with the one or more holes. In some embodiments, the shaft may define one lumen in fluid communication with each balloon 625, 627, 629. In some embodiments, the shaft 601 may define a first lumen in fluid communication with a subset of the holes corresponding to a first expandable electrode (e.g., electrode 605). and the shaft 601 may define a second lumen in fluid communication with a second subset of holes corresponding to the second expandable electrode (e.g., electrode 607). In some embodiments, the second lumen may also be in fluid communication with a third subset of holes corresponding to the third expandable electrode (e.g., electrode 609).

In FIG. 6, each electrode 605. 607 and 609 is shown in an expanded configuration. Thus, for example, the length 616 of the distal electrode 605 in this expanded configuration is reduced compared to the length 516 of the distal electrode 505 of FIG. 5 in its non-expanded state, while the diameter of the distal electrode 605 in this expanded configuration is larger compared to the length 516 of the distal electrode 505 of FIG. 5 in its non-expanded state. The separations or spacing 612, 614 between adjacent electrodes in the expanded configuration is larger compared to the respective separations or spacing 512, 514 in the non-expanded configuration shown in FIG. 5. For example, separation 612 measured between the (fixed) proximal end sleeve 640 of a first electrode 605 and the (sliding) distal end sleeve 644 of a second electrode in the expanded configuration is larger than the separation 512 between the first and second electrodes in the non-expanded configuration of FIG. 5. Likewise, separation 614 between the second electrode 607 and a third electrode 609 in the expanded configuration is larger than the corresponding separation 514 in the non-expanded configuration of FIG. 5. In embodiments, one or more of the electrodes can be indicated by a radio-opaque marker on the catheter shaft 601. For example, the catheter shaft 601 underneath the second electrode 607 can include a radio-opaque marker band 633 comprising radio-opaque material such as, for example, platinum, platinum-iridium alloy, tungsten, or other such materials known in the art that have high opacity to X-rays.

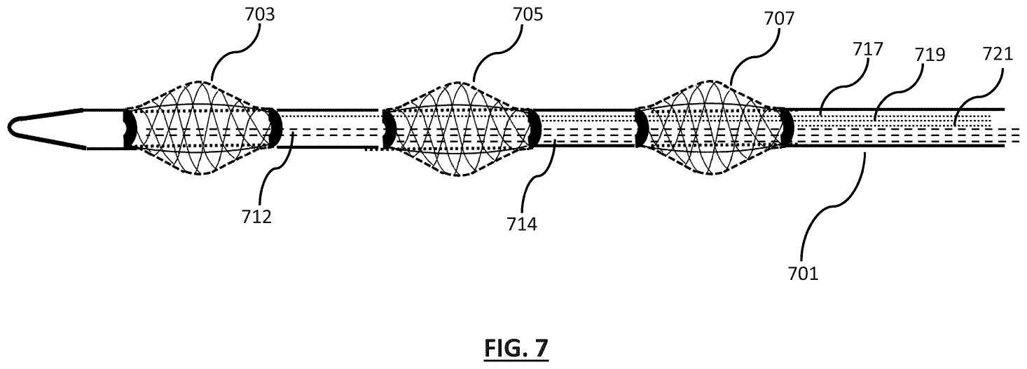

FIG. 7 illustrates an example catheter 701 of the present disclosure, showing in schematic form electrical leads connecting to the electrodes and lumens for balloon inflation to expand the electrodes, according to embodiments. The catheter 701 can be structurally and/or functionally similar to other catheters described herein, and include components that are structurally and/or functionally similar to those other catheters, such as those described with reference to the foregoing figures. For example, the catheter 701 can have three expandable electrodes 703, 705 and 707 that are constructed as described in the foregoing figures. In embodiments, an electrical lead wire 717 can connect to the first electrode 703, a second electrical lead wire 719 can connect to the second electrode 705, and a third electrical lead wire 721 can connect to the third electrode 707. In embodiments, an inflation lumen 712 connects to the balloon of the first electrode 703 for expanding the first electrode 703, while a second inflation lumen 714 connects to the balloon of electrodes 705 and 707 for expanding electrodes 705 and 707. While this provides a specific example of numbers of electrodes and inflation lumens, it should be clear that other numbers of electrodes can be utilized in the catheter construction and other numbers of inflation lumens can be used to address the inflation/deflation or activation of either single balloons or subsets of balloons, as convenient for the purpose at hand, without departing from the scope of the present invention.

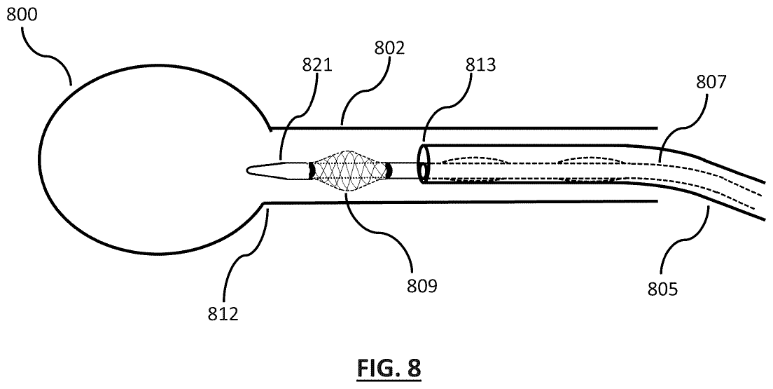

FIG. 8 provides a schematic illustration of an example catheter device of the present disclosure (e.g., any of the catheters depicted in FIGS. 5-7) used in conjunction with a cystoscope to access the prostatic urethra, according to embodiments. As shown, a cystoscope 805 is inserted into a urethra 802 of a subject or patient connecting to a bladder 800 in an anatomy of the subject or patient. In some embodiments, the catheter shaft 807 can be inserted through a working channel of the cystoscope 805, and forms the distal end portion 813 of the cystoscope 805. In some embodiments, the catheter distal end portion 821 can be placed near the bladder neck 812 such that the most distal electrode 809 is proximal to (e.g., immediately proximal to) the bladder neck 812 and is positioned distal to (e.g., immediately distal to) the distal end 813 of the cystoscope. The catheter 807 can be inserted and placed under visual guidance, e.g., using suitable imaging tools. For example, the cystoscope may capture image data distal to a distal end of the cystoscope and be coupleable to a display configured to display the image data to an user. This placement of the catheter device indicates engagement with the region of the prostatic urethra.

FIG. 9 shows an example catheter shaft 907 inserted through a working channel of cystoscope 905 disposed in the urethra 902 connecting to bladder 900 in the subject anatomy, according to embodiments. The catheter 907 can be structurally and/or functionally similar to other catheters described herein, including, for example, any of the catheter described with reference to FIGS. 5-8. Once a distal electrode 909 is positioned proximal to the bladder neck (e.g., similar to the placement relative to the bladder neck as described in FIG. 8), the corresponding balloon is inflated to deploy or expand the distal electrode 909 until it fully engages the wall of the urethra 902 and expands the wall radially outward so as to increase the internal diameter of the urethra 902. The balloon can be expanded, for example, by applying a suitable inflation pressure via a dedicated inflation lumen for the distal electrode 909. In some embodiments, the electrode may be expanded according to any method described herein. In embodiments, the balloon of at least the distal electrode 909 is able to sustain an inflation pressure of at least approximately 1.5 atmospheres. In this manner, the expanded distal electrode 909 expands the urethra 902 locally and is firmly pressed against the internal surface of the urethra 902. In this configuration, the catheter 907 is anchored in the urethra 902 to hold its position relative to the urethra 902.

FIG. 10 depicts an example catheter 1007 inserted through a working channel of a cystoscope 1005 disposed in the urethra 1002 connecting to a bladder 1000 in a subject anatomy, according to embodiments. The catheter 1007 can be structurally and/or functionally similar to other catheters described herein, including, for example, any of the catheter described with reference to FIGS. 5-9. In some embodiments, a distal electrode 1009 can be positioned proximal to a bladder neck similar to positioning described in FIGS. 8 and 9 and expanded via inflation to hold a constant position (e.g., to anchor or to stabilize the distal end of the catheter 1007) in the urethra 902 similar to the configuration and functionality discussed in FIG. 9. The cystoscope 1005 is then pulled back (e.g., withdrawn) while gently pulling on and holding the proximal portion (not shown) of the catheter 1007 (e.g., to fix a position of the catheter 1007 relative to the cystoscope 1005) so as to expose one or more electrodes proximal to the distal electrode, as needed for a length of prostate tissue surrounding the urethra 1002 that it is desired to treat. FIG. 10 shows a second electrode 1011 exposed in the urethra.

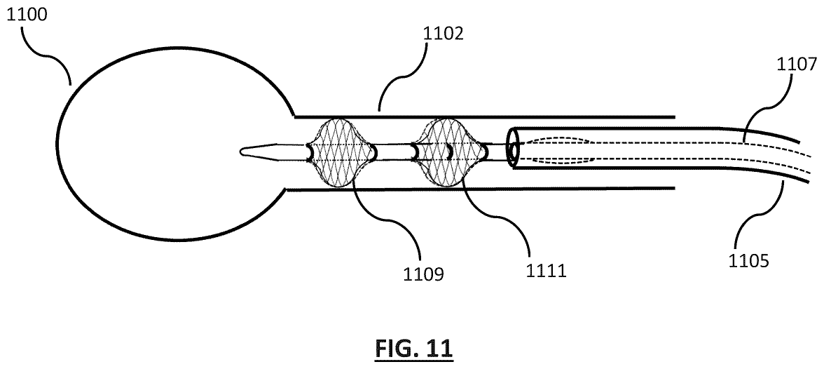

FIG. 11 illustrates an example catheter 1107 inserted through a working channel of a cystoscope 1105 disposed in a urethra 1102 connecting to a bladder 1100 in a subject anatomy. The catheter 1107 can be structurally and/or functionally similar to other catheters described herein, including, for example, any of the catheter described with reference to FIGS. 5-10. A distal electrode 1109 can be positioned proximal to the bladder neck and the distal electrode can be expanded (e.g., via inflation) to engage and expand the urethra (e.g., similar to that described in foregoing FIGS. 8, 9, and/or 10), and a second electrode 1111 that is exposed in the urethra 1102 can also be expanded (e.g., via inflation through a second inflation lumen) to engage and expand the urethra 1102, as shown in FIG. 11.

Once multiple electrodes 1109, 1111 engage the urethra 1102 (e.g., once multiple of the electrodes 1109, 1111 transition to the expanded configuration) as shown in FIG. 11, the electrodes 1109, 1111 can be electrically activated to deliver ablation. The electrode lead wires can be connected to a connector at the proximal end of the catheter (not shown), which in turn is coupled via a cable to a generator configured to deliver voltage waveforms for Pulsed Field Ablation (PFA). The generator can be configured to deliver a customized high voltage pulsed waveform comprising short-duration high voltage pulses for PFA. Such waveforms are described for example in International (PCT) Patent Application No. PCT/US2023/025064, titled “Apparatus. Systems and Methods for Soft Tissue Ablation,” filed June 12, 2023, and incorporated herein by reference. In embodiments, for PFA delivery, one subset of electrodes can be polarized with one electrical polarity while a second subset of electrodes can be polarized with the opposite electrical polarity. As an example, electrode 1109 can be electrically paired (opposite polarities) with electrode 1111 for bipolar ablation delivery. The voltage associated with the pulses can range from about 1 kV to about 10 kV, and all values and sub-ranges therebetween, depending on the pulse waveform and as appropriate to the procedure.

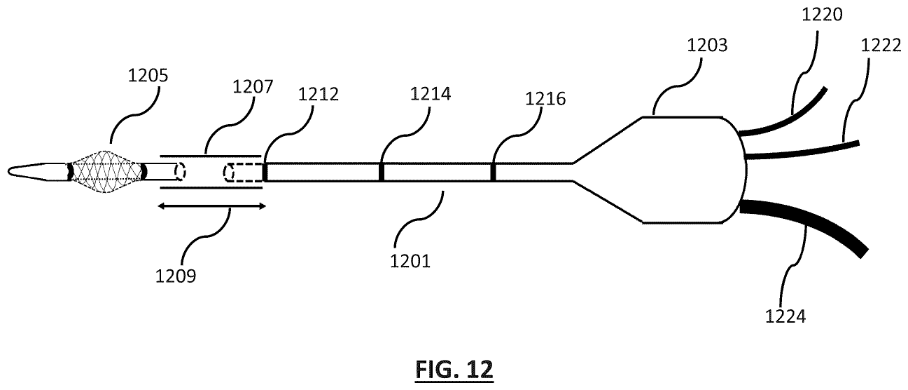

FIG. 12 schematically illustrates an example catheter of the present disclosure 1200 including its proximal portions, according to embodiments. The catheter can be structurally and/or functionally similar to other catheters described herein, including, for example, any of the catheter described with reference to FIGS. 5-11. A proximal shaft of the catheter terminates in a hub or handle 1203. In embodiments, there can be two inflation ports 1220 and 1222 attached to the hub 1203 and connecting to respective inflation lumens in the catheter. The inflation lumens are connected to distinct electrodes or electrode subsets. The ports 1220 and 1222 terminate in a standard Luer lock or valve (not shown) through which fluid can be infused, for example, from a syringe (not shown). An electrical cable 1224 terminating in an electrical connector (not shown) is used for connection to an extension cable (not shown), e.g., as needed for connection to a generator for high voltage pulse delivery for PFA. In embodiments, the proximal portion of the example catheter has markers 1212, 1214 and 1216 on the shaft. With the example catheter inserted through an entire working length 1209 of a cystoscope 1207 working channel (shown schematically as a short length in FIG. 12 for schematic illustrative purposes, which is not to scale), a distal electrode 1205 is outside (e.g., immediately outside) a distal end of cystoscope 1207. With this catheter positioning in the cystoscope, marker 1212 on the proximal catheter shaft is outside (e.g., immediately outside) the proximal end of the cystoscope working channel as shown in FIG. 12. Likewise, when the second electrode is outside (e.g., immediately outside) the distal end of cystoscope working channel 1207, marker 1214 would be outside (e.g., immediately outside) the proximal end of the cystoscope working channel, and when the third electrode is outside (e.g., immediately outside) the distal end of cystoscope working channel 1207, marker 1216 would be outside (e.g., immediately outside) the proximal end of the cystoscope working channel. In this manner, a visual check of the proximal section of the catheter shaft with markers can provide an indication of how far the catheter 1201 extends beyond the distal end of the cystoscope working channel 1207. Thus, the length of catheter shaft between the proximal end of the distal electrode and the distal marker 1212 corresponds to the length of the cystoscope working channel. In embodiments, an additional valve or other fluid port attachment can be included at the proximal end of the cystoscope working channel. In this case, the channel length of such an attachment is added to the cystoscope working channel length to determine placement of the distal marker 1212 on the catheter shaft.

FIG. 13 provides a schematic illustration of a proximal section of an example catheter 1300 of the present disclosure, showing markers 1315, 1317 and 1319 on the proximal portion of the catheter shaft, according to embodiments. The catheter can be structurally and/or functionally similar to other catheters described herein, including, for example, any of the catheter described with reference to FIGS. 5-12. The shaft terminates in a hub or handle 1302 that has an attached electrical cable 1312 (e.g., for high voltage delivery to the catheter electrodes) and an attached inflation port 1304. The catheter hub or handle 1302 can comprise an internal fluid manifold (not shown) for directing infused fluid to one or the other of two internal inflation lumens in the catheter 1300. In an embodiment, a switch or setting indicator 1306 can switch positions between markers 1308 and 1310 on the hub or handle 1302, corresponding to selection of the desired inflation fluid lumen for inflation of the appropriate balloon(s). The inflation port 1304 terminates in a standard Luer lock or valve (not shown) through which fluid can be infused, for example, from a syringe (not shown), and the setting indicator 1306 can be set at the appropriate marker 1308 or 1310 for inflation of the appropriate expandable electrodes at the distal portion of the catheter (not shown) via balloon inflation.

FIG. 14 provides a schematic illustration of an example catheter 1401 of the present disclosure that is passed through a working channel 1405 of a cystoscope showing a first electrode 1411 and a second electrode 1413 exposed outside the distal end of a cystoscope, according to embodiments. The catheter can be structurally and/or functionally similar to other catheters described herein, including, for example, any of the catheter described with reference to FIGS. 5-13. The proximal end of electrode 1413 is outside (e.g., immediately outside) the distal end of the cystoscope working channel 1405. A first marker 1422 located at a position on the catheter shaft (that takes into account the length 1460 of the working channel of the cystoscope and the first marker 1422) is visible outside (e.g., immediately outside) the proximal end of the cystoscope working channel, indicating that the second electrode 1413 is outside (e.g., immediately outside) the distal end of the cystoscope working channel. Marker 1424 is placed on the proximal catheter shaft such that the separation between marker 1422 and marker 1424 corresponds to the separation between the proximal end of the second electrode 1413 and the proximal end of a third electrode (not shown) proximal to the second electrode 1413 that is inside the cystoscope working channel. The catheter shaft terminates at a proximal handle or hub 1430 that is attached to two inflation fluid ports 1430 and 1432, and an electrical cable 1436 that is attached (possibly via an extension cable) to a console or generator 1441 (e.g., for high voltage pulse delivery for PFA). The respective inflation ports are used for deployment or inflation of corresponding balloon electrodes such as 1411 or 1413 in the distal portion of the catheter.

In embodiments, a display 1443 in the form of a touch screen or other type of monitor can be connected to the console 1441 and provides a user interface for delivery of pulsed electric field ablation therapy. The user interface comprises, among other visual elements, a voltage slider 1445 or other visual element for providing for selection of a voltage for ablation from a pre-defined range of voltages indicated on the voltage slider, and a selection menu 1453 or other visual element (for example, selection from a drop-down list) for selecting which electrodes are desired to be electrically activated for pulsed electric field ablation. An associated selection 1451 of the selected electrodes (for example, the distal two electrodes) is displayed on the user interface and changes as a different selection (for example, the distal three electrodes) is made.

The electrodes of the catheter of the present disclosure can have a length in the range between approximately 2 mm and approximately 50 mm in the undeployed or non-expanded state, including all sub-ranges and values therebetween. In embodiments, upon inflation of the underlying balloon, the electrode length is reduced by at least about 10%. The spacings (nearest edges) between adjacent electrodes can lie in the range between about 1 mm and about 40 mm, including all sub-ranges and values therebetween. In embodiments, upon inflation of the underlying balloon, the distance between adjacent electrodes can increase by at least about 20%

In embodiments, the diameter (i.e., largest width transverse to the longitudinal axis or length) of the expandable electrodes in the non-expanded state can lie in the range of between approximately 1 mm and approximately 10 mm, including all sub-ranges and values therebetween. In embodiments, the diameter of the expandable electrodes in the fully expanded state can lie in the range of between approximately 7 mm and approximately 30 mm, including all sub-ranges and values therebetween. In embodiments, one or more radio-opaque markers can be associated with at least one of the electrodes, e.g., as indicated by marker 633 in FIG. 6.

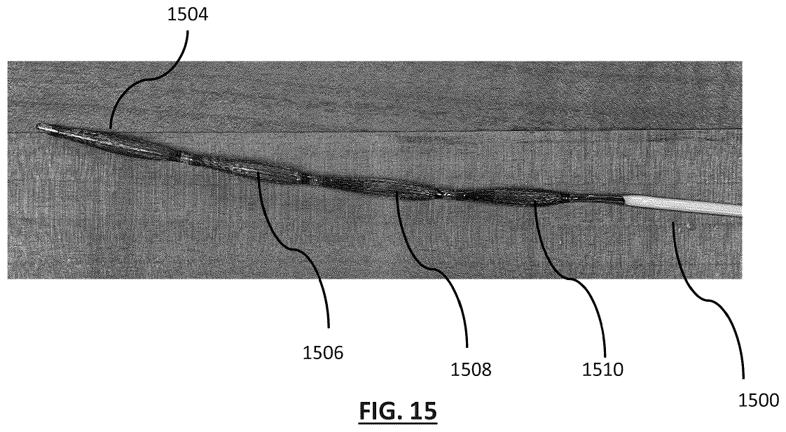

While specific examples such as the number of electrodes (e.g., three in the drawings or figures) have been provided in this disclosure and attached figures, it should be clear that catheters with other numbers of electrodes can be built and deployed according to the teachings herein without departing from the scope of the invention. For example, FIG. 15 provides an illustration of a catheter 1500 with four expandable electrodes 1504, 1506, 1508 and 1510 with electrodes in the undeployed state.

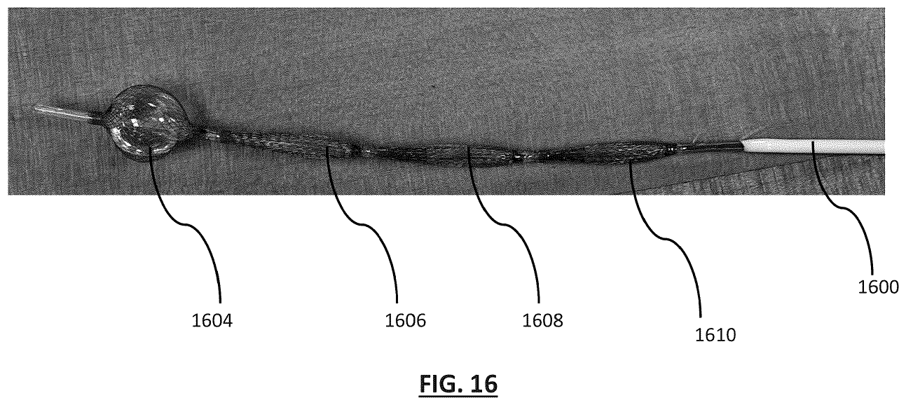

FIG. 16 illustrates a catheter 1600 with four expandable electrodes 1604, 1606, 1608 and 1610 with a distal electrode 1604 deployed or expanded by inflation of its balloon, while the other three electrodes 1606, 1608 and 1610 are in the undeployed state.

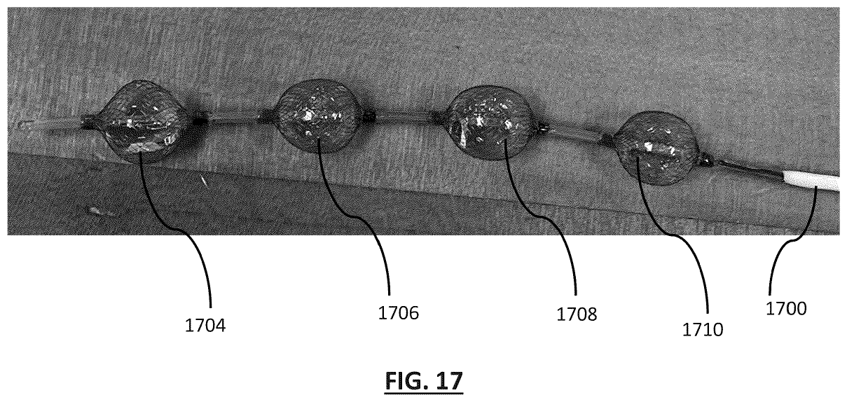

FIG. 17 illustrates a catheter 1700 with four expandable electrodes with all four electrodes 1704, 1706, 1708 and 1710 expanded by inflation of corresponding balloons. While the diameter of each electrode has increased due to inflation, each electrode length is decreased compared to the non-expanded configuration of FIG. 15, while separation between adjacent electrodes has increased due to sliding of the distal end sleeve of each electrode on the catheter shaft.

In alternate embodiments, other numbers of expandable electrodes can be used, ranging from 1 to 12, including all sub-ranges and values therebetween, according to a convenience or the requirements of a given application. In the case of a single expandable electrode being used, a reference patch electrode placed externally on the surface of the subject anatomy is electrically paired with the single expandable electrode for pulsed electric field ablation. Likewise, while two inflation lumens for inflation of electrodes are discussed in the examples herein, other numbers of inflation lumens, ranging from 1 to 8 inflation lumens, including all sub-ranges and values therebetween, can be disposed internally in the catheter shaft for inflation of various subsets of electrodes.

In embodiments, each balloon that is utilized to expand an electrode can be inflated to a pressure of at least approximately 1.5 atmospheres or greater without rupture. In other embodiments, each balloon can be inflated to a pressure of at least approximately 2.5 atmospheres or greater without rupture.

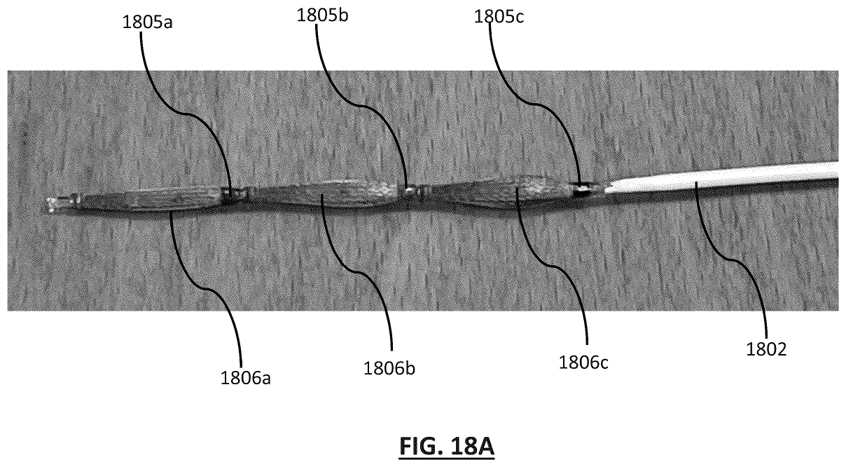

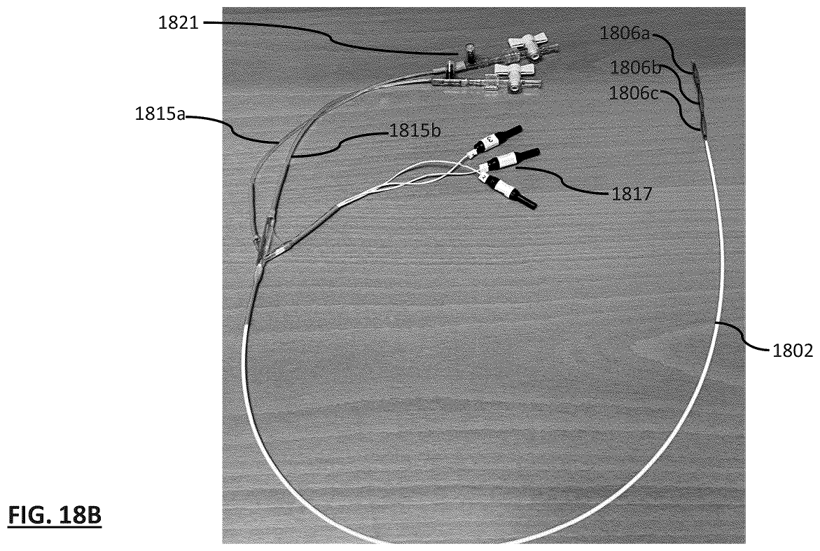

FIGS. 18A-18B illustrates a catheter shaft 1802 including a first electrode 1806a, a second electrode 1806b, and a third electrode 1806c disposed along a distal end of the shaft 1802. The shaft 1802 further includes a first marker 1805a disposed at a proximal end of the first electrode 1806a, a second marker 1805b disposed at a proximal end of the second electrode 1806b, and a third marker 1805c disposed at a proximal end of the third electrode 1806c. In embodiments, the color of each marker is distinct. For example, the first marker 1805a can be a blue marker, whereas the second marker 1805b can be a yellow marker, and the third marker 1805c can be black in color. Therefore, the first marker 1805a is easily distinguishable from the second marker 1805b and third marker 1805c during the procedure such that the user can distinguish the first electrode 1805a (e.g., the distal electrode) from each of the proximal electrodes. In some embodiments, a distal end of each electrode (or other portions of the electrode) may optionally include a marker configured to indicate when the distal end of the electrode is disposed distal to the cystoscope.

The shaft 1802 can define a first lumen (not shown) in fluid communication with the first electrode 1806a, and second lumen (not shown) in fluid communication with the second electrode 1806b and third electrode 1806c. As shown in FIG. 18B, catheter shaft 1802 can incorporate two separate channels 1815a, 1815b each configured to be coupled to a fluid source. The channels 1815a, 1815b can be coupled to the fluid source via ports (e.g., Luer Locks) at a proximal end thereof. In some embodiments, the first channel 1815a can be configured to supply fluid through to first lumen and to the first electrode 1806a. The second channel 1815b can be configured to supply fluid to the second lumen and to the second electrode 1806b and the third electrode 1806c. In embodiments, the fluid line can include a pressure relief valve, for example, a pressure relief valve 1821 attached to fluid line 1815a. The pressure relief valve 1821 can be configured to limit pressure to a pre-determined level, for example between about 0.4 atmosphere (atm) and about 10 atm (including, for example, about 2 atm), such that it leaks and releases fluid through a port in the valve if more fluid infusion is attempted to inflate the already inflated balloon. This prevents over-inflation of the balloon and can prevent the balloon from bursting due to over-inflation. In embodiments, both or all fluid lines can incorporate such a pressure relief valve. The pre-determined pressure level of the pressure relief valve can lie in the range between approximately 0.4 atm and approximately 10 atm, including all values and sub-ranges therebetween. The proximal end of the shaft 1802 can further be coupled to one or more electrical connectors 1817. The electrical connectors 1817 can be configured to couple the electrodes 1806a, 1806b, 1806c to a pulse generator. Each electrode 1806a, 1806b, 1806c may be coupled to a respective electrical connector, or a distinct pin on a single multi-port electrical connector, via a wire extending along the shaft 1802. Therefore, each electrode 1806a, 1806b, 1806c can be independently electrically controlled by the pulse generator to generate the desired electric field. The catheter shaft can be structurally and/or functionally similar other catheters described herein, including, for example, any of the catheter described with reference to FIGS. 1-13.

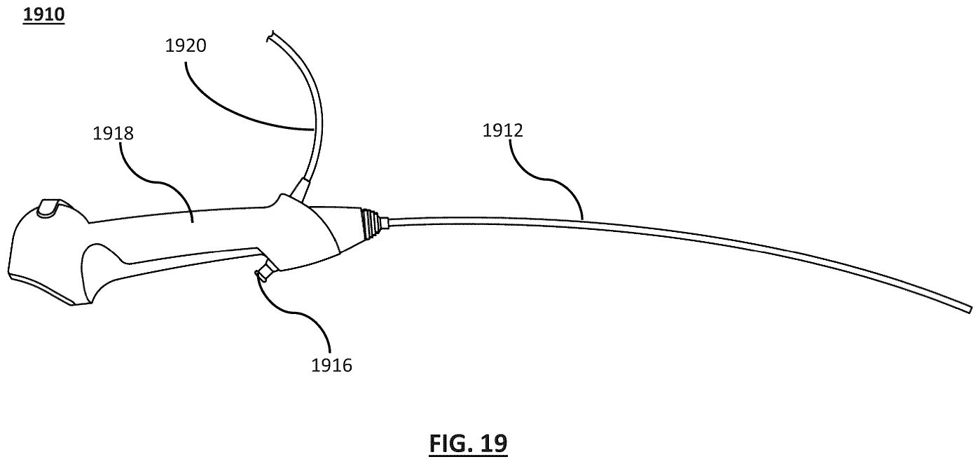

FIG. 19 illustrates an example cystoscope 1910 configured to receive a catheter shaft (e.g., any of the shafts described herein) for delivering pulsed field ablation device in a patient. As shown, the cystoscope 1910 can include a handle 1918 configured to be engaged by the user to navigate the insertion tube or cystoscope shaft 1912 through the anatomy of the patient. The tube 1912 may define a working channel coupled to port 1916 on the handle through which the catheter shaft can be disposed. In some embodiments, the cystoscope 1910 includes an imaging element (not shown). The handle 1918 may include a connector 1920 configured to transmit signals corresponding to image data captured by the imaging element to an external device such as a compute device or display.

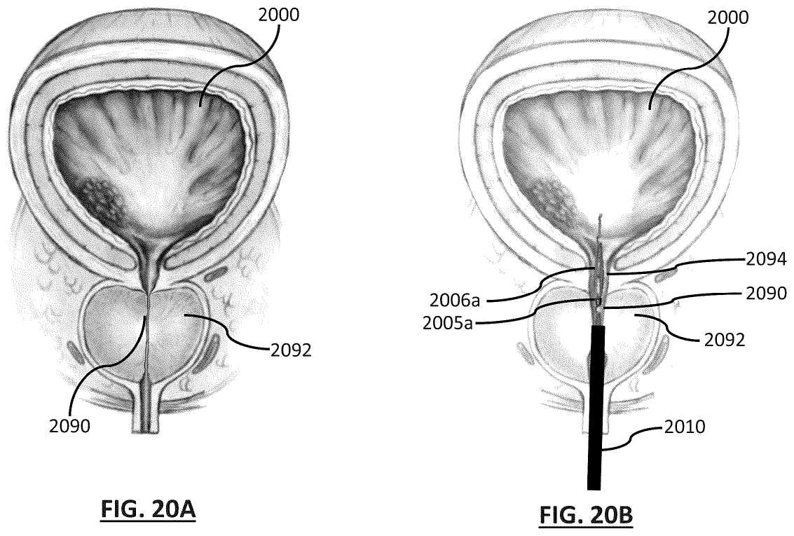

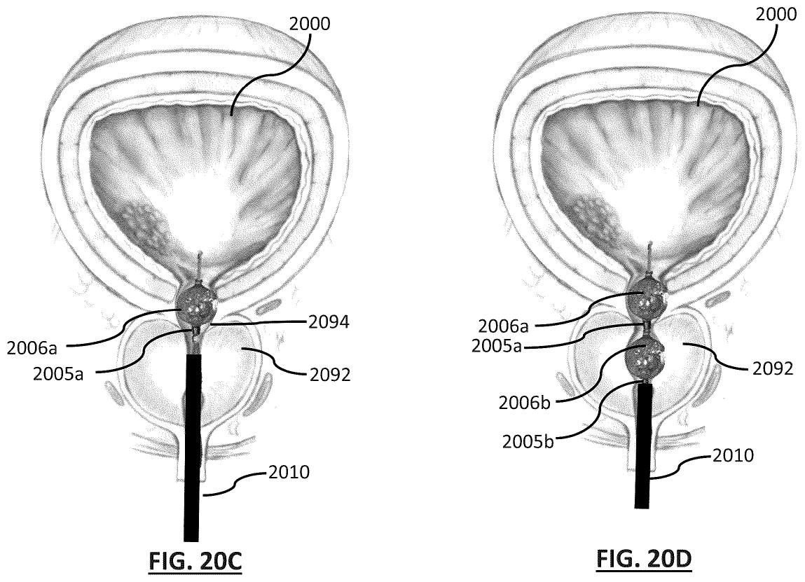

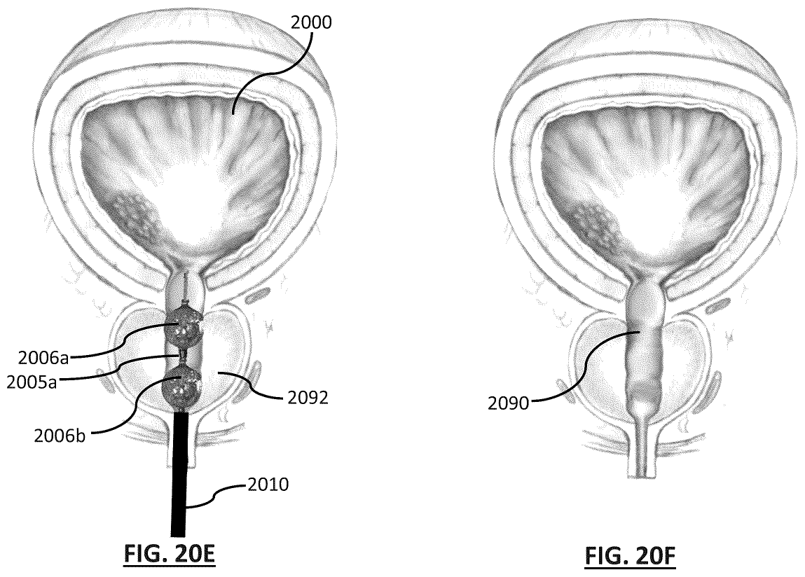

FIGS. 20A-20F show a method of positioning electrodes relative to a prostatic urethra to deliver pulsed field ablation to treat BPH. FIG. 20A is an illustration of a bladder 2000, and an enlarged prostate 2092 causing obstruction of the urethra 2090. As shown in FIG. 20B, a tube or 2010 (e.g., shaft of a cystoscope) can be navigated through the urethra 2090 adjacent to the prostate 2092. The tube 2010 may be positioned such that a distal end of the tube 2010 is proximal to or is situated at a bladder neck 2094 of the patient. A catheter shaft slidably disposed through the tube 2010 can be advanced distally through the tube 2010 such that a first electrode 2006a is disposed distal to the distal end of the tube 2010 and positioned within the bladder neck 2094 (e.g., an anchor location) and a distal end of the shaft is disposed in the bladder 2000. In some embodiments, the first electrode 2006a can be advanced until a first marker 2005a associated with the first electrode 2006a is visible to indicate the first electrode 2006a is disposed distal to the distal end of the tube 2010.

As shown in FIG. 20C, the first electrode 2006a can be transitioned from the unexpanded configuration to an expanded configuration to engage and expand the bladder neck 2006a to anchor the distal portion of the shaft relative to the urethra and the prostate. As shown in FIG. 20D. the tube or cystoscope shaft 2010 can be withdrawn proximally or retracted to expose a second electrode 2006b. For example, the tube 2010 can be withdrawn proximally until a second marker 2005b associated with the second electrode 2006b is visible indicating the second electrode is disposed distal to the distal end of the tube 2010. The second electrode 2006 can then be transitioned from the unexpanded configuration to the expanded configuration to engage and expand the wall of the urethra. After both the first electrode 2006a and the second electrode 2006b are transitioned to the expanded configuration, pulsed field ablation can be applied via the first electrode and the second electrode to ablate prostate 2092 around the urethra.

As shown in FIG. 20E, after pulsed field ablation is applied to a first portion of target tissue, the distal portion of the shaft can be repositioned. For example, the first electrode 2006a and the second electrode 2006b can be transitioned to the unexpanded configuration to allow the shaft to be moved relative to the target tissue. Once repositioned, the first electrode 2006a and the second electrode can be transitioned to the expanded configuration and activated to apply pulsed field ablation to a second portion of the target tissue. FIG. 20F shows the urethra 2090 following pulsed field ablation.

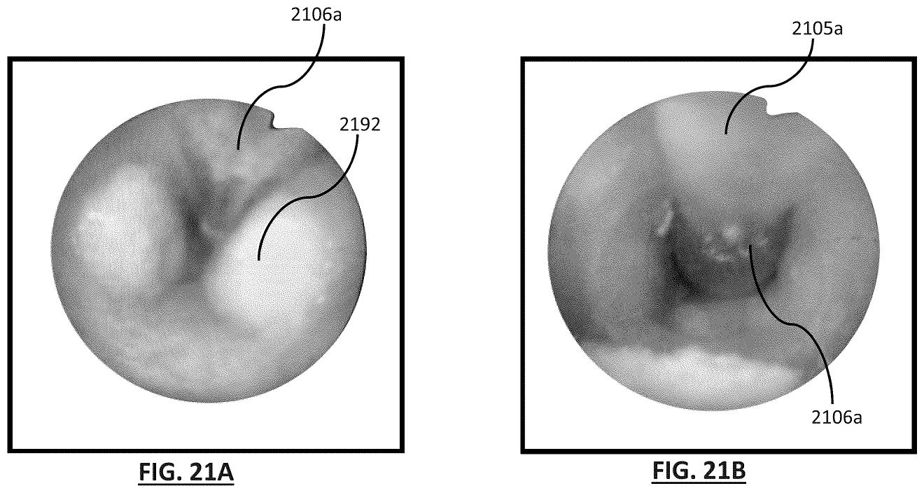

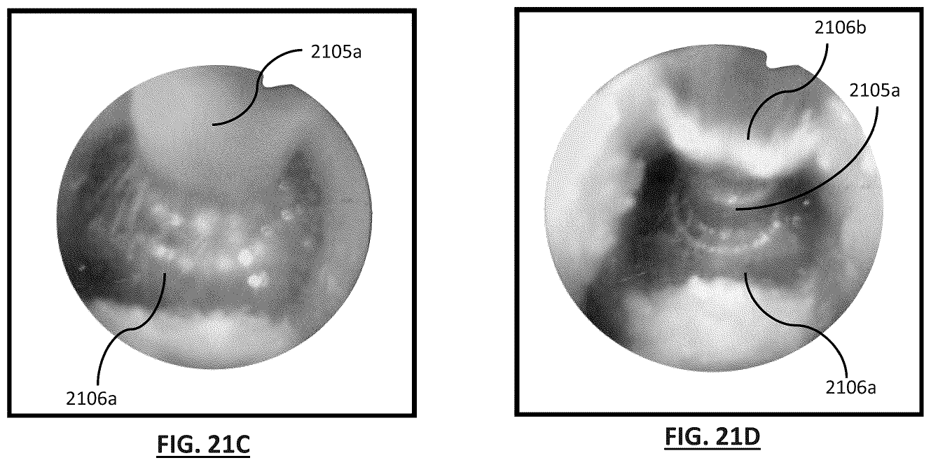

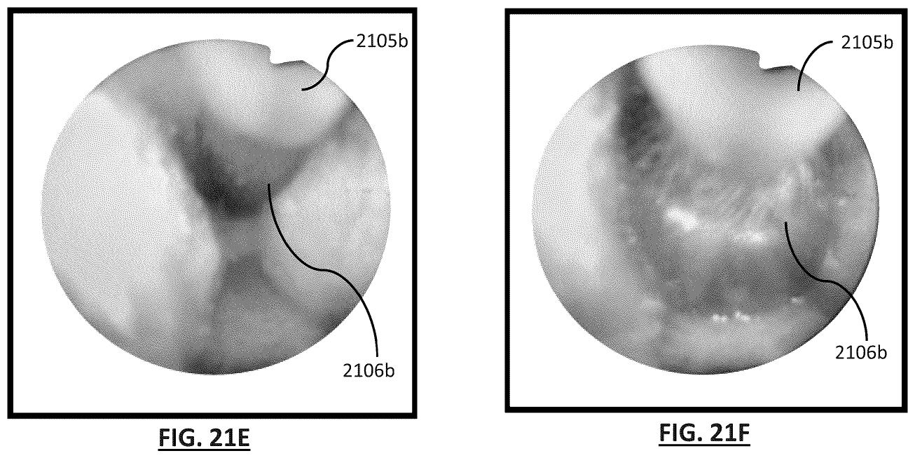

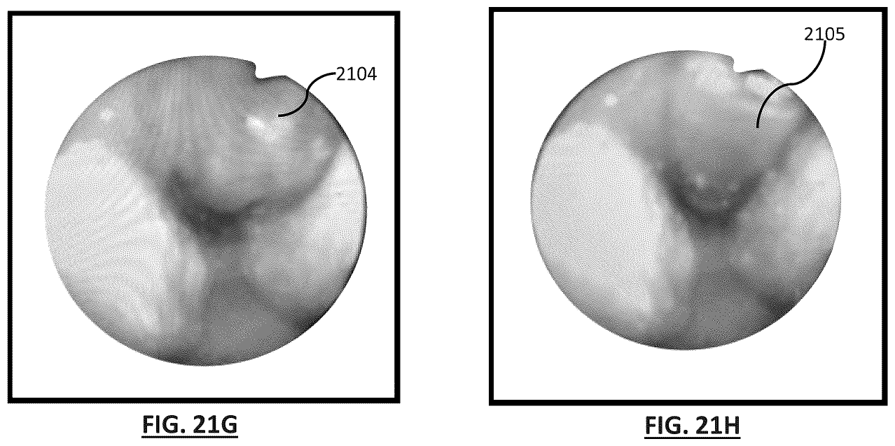

FIGS. 21A-21H show cystoscopic images of a procedure for delivering pulsed field ablation to a prostate, according to embodiments. As shown in FIG. 21A, a cystoscope can be positioned at the start of the prostatic urethra near the apex of the prostate (e.g., with prostate lobes such as lobe 2192 being visible in the image) with a distal end of a distal electrode 2106a visible on camera. As shown in FIG. 21B, the cystoscope can be advanced toward a position just proximal to a bladder neck of the patient. The first electrode 2016a can be advanced distally until a first marker 2015a (shown in blue) is visible on camera. As shown in FIG. 21C, a first expandable member (e.g., balloon) can be inflated to expand the first electrode against the bladder neck. In some embodiments, the first expandable member may be inflated until a pressure relief valve at a proximal end of the shaft starts to leak (e.g., indicating the expandable member is contacting the bladder wall with a predetermined force). In some embodiments, the first electrode 2105 expanded against the bladder neck holders the shaft in a stable position relative to the urethra. As shown in FIG. 21D, the catheter can be held (e.g., fixed) at a proximal end while the cystoscope is retracted proximally over the distal portion of the shaft such that a second electrode 2106b is disposed distal to the tube of the cystoscope.

As shown in FIG. 21E, the cystoscope can be retracted until a second marker 2105b (shown in yellow) associated with the second electrode 2106b is visible, indicating the second electrode is fully disposed distal to the cystoscope. As shown in FIG. 21F, a second expandable member (e.g., balloon) can be inflated to expand the second electrode against the wall of the urethra. In embodiments, the second expandable member can be inflated until the pressure relief valve starts to leak. Although shown with two electrodes, it should be appreciated any suitable number of electrodes can be expanded along a length of the urethra depending on the anatomy. As shown in FIG. 21G, in some procedures the expandable members can be deflated, and the catheter and cystoscope can be withdrawn proximally (e.g., about 0.5 cm, about 1 cm, about 1.5 cm, about 2 cm, about 2.5 cm, about 3 cm, between about 0.5 cm and about 5 cm, etc., inclusive of all sub-ranges and values therebetween). Once repositioned, the electrodes can be transitioned to the expanded configuration and pulsed field ablation can be delivered to a second portion of tissue. Once the target tissue is sufficiently ablated, the electrodes can be transitioned to the unexpanded configuration, and the cystoscope and catheter can be withdrawn out of the patient.

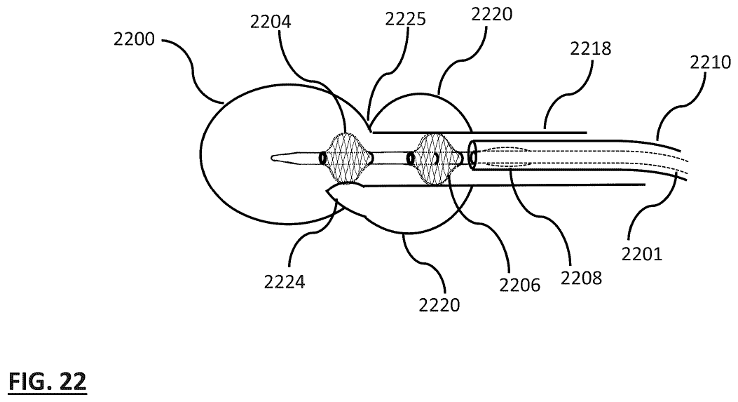

FIG. 22 illustrates a cystoscope 2210 in a urethra 2218 that terminates in bladder 2200. Catheter 2201 extends through the distal end of the cystoscope 2210. Inflatable electrodes 2204, 2206 on the distal portion of a shaft of the catheter 2201 extend beyond the distal end of the cystoscope 2210 and are deployed in the bladder 2200 and urethra 2218 respectively. In some cases, the prostate 2220) surrounding the urethra has a median lobe 2224 that protrudes into the bladder 2200. In some embodiments, the distal electrode 2204 can be positioned to abut the median lobe 2224. For example, the distal electrode 2204 can be deployed beyond the bladder neck 2225 into the bladder 2220) so as to abut the median lobe 2224. When the electrodes 2204, 2206 are activated for delivery of pulsed field ablation therapy, therapy is delivered to the prostate 2220, including the median lobe 2224. In this manner the entire prostate 2220), including the median lobe, can be treated with PFA therapy.

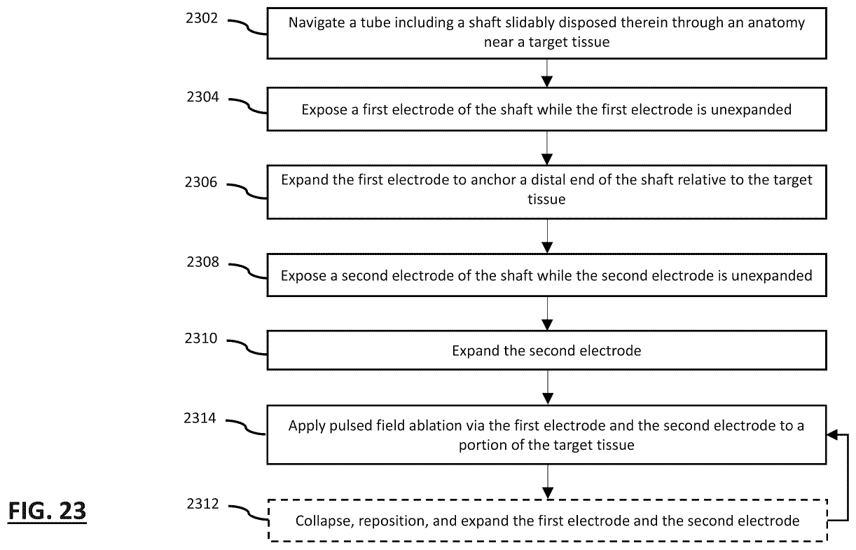

FIG. 23 a is flow chart of an example method of delivering ablative energy to a target tissue, according to embodiments. The example method can be implemented using any cystoscope or catheter described herein in FIGS. 1-22. In some embodiments, the method can include navigating a tube including a catheter shaft slidably disposed therein through an anatomy (e.g., a body lumen) of a patient near a target tissue, at 2302. The method may include exposing a first electrode of the shaft while the first electrode is unexpanded in the anatomy of the patient, at 2304. In some embodiments, the shaft may be advanced distally to dispose the first electrode distal to the tube. In some embodiments, the method may include expanding the first electrode to anchor a distal end of the shaft relative to the target tissue, 2306. For example, the first electrode may include an expandable element and a conductive element. In some embodiments, the expanding the first electrode can include conveying fluid via a first lumen to the expandable element of the first electrode such that the expandable element inflates and causes the conductive element to expand. In some embodiments, the expanding the first lumen may convey fluid to the expandable element of the first electrode and not the expandable member of the second electrode such that only the first electrode expands.

In some embodiments, the method may include exposing a second electrode of the shaft while the second electrode is unexpanded, at 2308. In some embodiments, the tube may be withdrawn to expose the second electrode. In some embodiments, the method may include expanding the second electrode, at 2310. The second electrode may be expanded to engage and expand a portion of the anatomy (e.g., the body lumen) proximal to the first electrode. In some embodiments, the second electrode may include an expandable element and a conductive element. In some embodiments, the expanding the second electrode can include conveying fluid through a second lumen separate from the first lumen to the expandable element of the second electrode such that the expandable element inflates and causes the conductive element to expand.

In some embodiments, the method may include applying pulsed field ablation via the first electrode and the second electrode (e.g., when the first electrode and the second electrode are expanded) to a portion of the target tissue, at 2314. In some embodiments, the method may include collapsing (e.g., transitioning to an unexpanded configuration), repositioning, and expanding the first electrode and the second electrode to reposition the distal portion of the shaft, at 2312. In some embodiments, the applying the pulsed field ablation, at 2314, and the repositioning, at 2312 can be repeated until the desired target tissue is ablated and/or the body lumen is unobstructed.

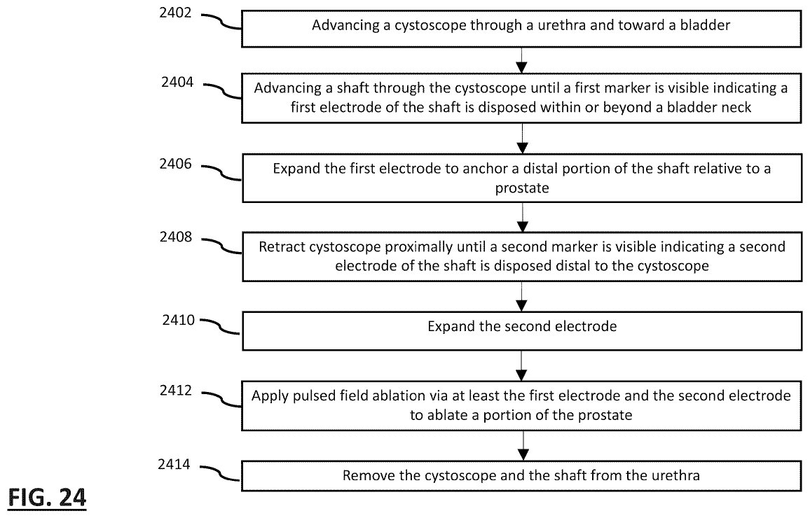

FIG. 24 is a flow chart of an example method of positioning a catheter shaft including expandable electrodes relative to a prostate, according to embodiments. The example method can be implemented using any of the cystoscopes or catheters described herein in FIGS. 1-22. In some embodiments, the method may include advancing a cystoscope through a urethra. at 2402. In some embodiments, the advancing the cystoscope may include navigating the distal end of the cystoscope to a bladder neck or bladder of a patient. In some embodiments, the method may include advancing a shaft through a working channel of the cystoscope until a first marker associated with a first electrode is visible on the cystoscope images, indicating the first electrode of the shaft is disposed within or beyond the bladder neck, at 2404. In some embodiments, the method may include expanding the first electrode to anchor the distal portion of the shaft relative to a prostate, at 2406. In some embodiments, the method may include retracting the cystoscope proximally until a second marker associated with a second electrode is visible, indicating a second electrode of the shaft is exposed (e.g., disposed distal to the cystoscope), at 2408. In some embodiments, the method can include expanding the second electrode 2410. The second electrode 2410 is expanded after the second marker is visible. In some embodiments, the method can include applying pulsed field ablation via at least the first electrode and the second electrode to ablate a portion of the prostate, at 2412. In some embodiments, the method may further include repositioning the first electrode and the second electrode in an unexpanded configuration relative to the wall of the urethra and the prostate and expanding the first electrode and the second electrode against a second portion of the wall of the urethra. The method may include applying pulsed field ablation to the second portion of the urethra and/or prostate. The method may include removing the cystoscope and the shaft from the urethra once the prostate is sufficiently treated, at 2414.

While specific examples have been provided in the disclosure for exemplary purposes, variations can be conceived without departing from the scope of the present disclosure. Likewise, while the prostatic urethra has been discussed as an example clinical application in this disclosure, it should be apparent that a device embodiment can be used in other anatomical passageways. For example, an ablation device according to the present disclosure can be used with an endoscope instead of a cystoscope for ablation applications along the digestive tract, for example, for ablation of the esophagus.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

1. An apparatus, comprising:
a shaft configured to be navigated through an anatomy toward a target tissue of a patient; and
a plurality of electrodes disposed on a distal portion of the shaft and spaced axially along the shaft, the plurality of electrodes including a distal electrode and a proximal set of electrodes, the distal electrode configured to transition from a unexpanded configuration to an expanded configuration independently from the proximal set of electrodes to anchor the distal portion of the shaft relative to the target tissue, the proximal set of electrodes configured to transition from a unexpanded configuration to an expanded configuration to engage and expand neighboring tissue,
the plurality of electrodes, after the distal electrode and the proximal set of electrodes are in the expanded configuration, being configured to deliver pulsed field ablation to the target tissue.

2. The apparatus of claim 1, wherein each electrode from the plurality of electrodes includes an expandable element and a conductive element.

3. The apparatus of claim 2, wherein the shaft defines one or more lumens, the expandable elements of the plurality of electrodes being in fluid communication with the one or more lumens.

4. The apparatus of claim 1, wherein each electrode from the plurality of electrodes includes a cylindrical braid formed from a plurality of conductive strands.

5. The apparatus of claim 1, wherein each electrode from the plurality of electrodes has a first end that is fixed to the shaft and a second end that is configured to move along the shaft as the electrode is transitioned from the unexpanded configuration to the expanded configuration.

6. The apparatus of claim 1, wherein the target tissue is a prostate of the patient, and the plurality of electrodes are configured to engage and expand a wall of the urethra.

7. The apparatus of claim 1, wherein the shaft further includes a first marker and a second set of markers, the first marker disposed at a proximal end of the distal electrode, each marker from the second set of markers disposed at a proximal end of an electrode from the proximal set of electrodes.

8. The apparatus of claim 7, wherein each of the first marker or the second set of markers is of a distinct color.

9. An apparatus, comprising:
a shaft configured be disposed near a target tissue of a patient, the shaft defining a first lumen and a second lumen along a length thereof;
a first electrode including a first conductive element and a first expandable element, the first lumen configured to convey fluid to the first expandable element to expand the first expandable element such that the first conductive element transitions from an unexpanded configuration to an expanded configuration; and
a second electrode including a second conductive element and a second expandable element, the second lumen configured to provide fluid to expand the second expandable element such that the second conductive element transitions from an unexpanded configuration to an expanded configuration,
at least one of the first electrode or the second electrode in the expanded configuration configured to engage and expand neighboring tissue, and the first electrode and the second electrode configured to deliver pulsed field ablation to the target tissue.

10. The apparatus of claim 9, wherein the first lumen terminates in a first opening defined by the distal portion of the shaft and the second lumen terminates in a second opening defined by the distal portion of the shaft.

11. The apparatus of claim 10, wherein the first expandable element is disposed around the first opening, and the second expandable element is disposed around the second opening.

12. The apparatus of claim 9, wherein the first expandable element and the second expandable element are inflatable balloons.

13. The apparatus of claim 9, further comprising:
a third electrode including a third conductive element and a third expandable element, the second lumen configured to convey fluid to the third expandable element to expand the third expandable element such that the third conductive element transitions from an unexpanded configuration to an expanded configuration.

14. The apparatus of claim 9, wherein a proximal end of the shaft is coupled to a flow control mechanism including a first configuration in which fluid is allowed to flow through the first lumen and is prevented from flowing through the second lumen

15. The apparatus of claim 14, wherein the flow control mechanism includes a second configuration in which fluid is allowed to flow through the second lumen.

16. The apparatus of claim 9, wherein the first electrode and the second electrode each include a cylindrical braid formed from a plurality of conductive strands.

17. The apparatus of claim 9, wherein the target tissue is a prostate of the patient, and at least one of the first electrode or the second electrode is configured to engage and expand a wall of the urethra

18. An apparatus, comprising:
a shaft configured to be navigated through a working channel of a cystoscope to a urethra of a patient;
a plurality of electrodes spaced axially along a distal portion of the shaft; and
a plurality of markers, each marker from the plurality of markers disposed at a proximal end of a respective electrode from the plurality of electrodes to indicate when the respective electrode is disposed distal to a distal end of the cystoscope,
the plurality of electrodes configured to transition from an unexpanded configuration to an expanded configuration in which at least one of the plurality of electrodes is configured to engage and expand a wall of the urethra and the plurality of electrodes are configured to deliver pulsed field ablation energy to at least a portion of the urethra and prostate tissue adjacent thereto.

19. The apparatus of claim 18, wherein the plurality of electrodes includes a distal electrode and a proximal electrode.

20. The apparatus of claim 19, wherein the plurality of markers includes a first marker disposed at a proximal end of the distal electrode and a second marker disposed at a proximal end of the proximal electrode.

21. The apparatus of claim 20, wherein the first marker is visually different from the second marker.

22. The apparatus of claim 18, wherein the plurality of markers include colored markers.

23. The apparatus of claim 18, wherein each electrode from the plurality of electrodes includes a cylindrical braid formed from a plurality of conductive strands.

24. A method, comprising:
navigating a shaft to an anatomy of a patient toward a target tissue, the shaft including a plurality of electrodes disposed on a distal portion of the shaft and spaced axially along the shaft;
expanding a distal electrode from the plurality of electrodes against a portion of tissue near the target tissue to anchor the distal portion of the shaft relative to the target tissue;
expanding, after expanding the distal electrode, a proximal set of electrodes of the plurality of electrodes; and
applying, after the distal electrode and the proximal set of electrodes are expanded, pulsed field ablation to at least a portion of the target tissue via the plurality of electrodes.

25. The method of claim 24, wherein the navigating the shaft to the anatomy of the patient includes:
disposing a visualizing scope in the anatomy and advancing the shaft through a working channel of the visualizing scope adjacent to the target tissue.

26. The method of claim 25, wherein the method further includes:
advancing, before expanding the distal electrode, the distal electrode distal to a distal end of the visualizing scope such that the distal electrode is adjacent to the target tissue.

27. The method of claim 25, wherein the method further includes:
withdrawing, before expanding the proximal set of electrodes, the visualizing scope proximally, with the distal electrode held fixed, such that the proximal set of electrodes is disposed distal to the visualizing scope.

28. The method of claim 24, wherein the expanding the distal electrode from the plurality of electrodes includes conveying fluid from a fluid source through a lumen of the shaft to an expandable element of the distal electrode, and expanding the proximal set of electrodes includes conveying fluid through a second lumen of the shaft separate from the first lumen to expandable elements of the proximal set of electrodes.

29. The method of claim 24, further comprising:
transitioning the proximal electrode and the distal electrode from an expanded configuration to an unexpanded configuration; and
repositioning the proximal electrode and the distal electrode relative to the target tissue.

30. The method of claim 24, wherein the target tissue is a prostate, the expanding the distal electrode includes:
expanding the distal electrode to engage and expand at least a portion of a bladder neck or engage a proximal portion of the bladder to anchor the distal portion of the shaft relative to the prostate.
